(19) United States
(12) Patent Application Publication
(10) Pub. No.: US 2019/0022667 A1
(43) Pub. Date: Jan. 24, 2019

(54) MULTIFUNCTIONAL BIOLOGICAL SUBSTANCE SEPARATION DEVICE

(71) Applicants: SHANXI ZDGSY BIO-SCIENTIFIC CO., LTD., Shanxi (CN); SHANXI UNIVERSITY OF CHINESE MEDICINE, Yuci, Shanxl (CN)

(72) Inventors: Guisheng Yang, Toronto (CA); Junsen Yang, Lvliang City (CN)

(21) Appl. No.: 16/071,038

(22) PCT Filed: Aug. 5, 2016
(86) PCT No.: PCT/CA2016/000204

§ 371 Date: Jul. 18, 2018

(30) Foreign Application Priority Data
Jan. 19, 2016 (CN) 201610033982.3
Jan. 19, 2016 (CN) 201610034026.7
Jan. 19, 2016 (CN) 201620049624.7

(51) Int. Cl. B03C 1/28; C12N 15/10; C12M 1/42

(52) U.S. Cl. CPC B03C 1/286; C12N 15/1013; B03C 2201/26; B03C 2201/18; C12M 1/42

(57) ABSTRACT

The present invention provides a multifunctional biological substance separation device or dehydration device illustratively comprising a housing, a spindle push rod running through the housing, a return device, a magnetic sleeve connecting ring, and an electric actuator. An end of the spindle push rod is provided with a magnetic column. The magnetic sleeve connecting ring is provided on the spindle push rod in a sleeving manner and one end, which is positioned outside the housing, of the connecting ring is provided with a magnetic sleeve. The return device is provided in the housing and is used for enabling the spindle push rod to move in a direction away from the magnetic sleeve. The electric actuator is connected to the connecting ring and is used for enabling the connecting ring (and the magnetic sleeve provided thereon) to rotate.

### FIELD OF THE INVENTION

The present invention relates to the fields of biology and biotechnology and to devices and methods for the separation of substances, including biological substances, and more particularly to multi-functional devices, including the functions of dehydration, and a sleeve to protect the magnet.

### BACKGROUND OF THE INVENTION

In the technical fields of biology and biotechnology, for the separation of biological substances, the main operations that are currently undertaken are centrifugation, filtration, extraction, electrophoresis, etc. The principle of these methods is to rely on the differences in respective weights and size diameters to separate different solid substances. For example, substance separation by using a centrifuge relies on the differences in the respective masses (specific gravity) of the solid or liquid substances in a liquid and producing different interfaces under the action of centrifugal force to achieve the purpose of substance separation. Filtration (including sieving and screening) relies on the aperture diameter of the filter membrane and the diameter of the filterable substance to achieve the purpose of substance separation. Drying, volatilization, extraction, electrophoresis and other methods all rely on the physical and/or chemical properties of the substances to achieve the result of substance separation.

For the above traditional methods of separation, particularly, the application of the centrifugal method in biological substance separation, the disadvantage is that a centrifuge tube must be marked clearly before centrifugation each time, otherwise a sample label cannot be determined after centrifugation. Marking a centrifuge tube is time-consuming, particularly when dealing with a large number of samples. in addition, the centrifugal method can only roughly separate substances of the same mass to a certain level (interface), but cannot separate substances of the same mass but having different biological activity.

Filtration, extraction, electrophoresis, and the like all have the disadvantage that substances of the same size or mass can only be separated to a certain level but substances of the same mass but different biological activity cannot be separated. For the equipment used in filtration, extraction, electrophoresis, and the like, the operation processes are cumbersome, sample tubes are easy to be marked incorrectly, and aseptic operation is difficult to carry out. The accuracy of substance separation is difficult to control and substances cannot be distinguished according to their biological activity.

In recent years, in processes for the separation of cells, viruses, proteins, nucleic acids etc., the principles of immunology and nanometer magnetic beads have been applied, such as a magnetic frame/stand methodology and automatic extraction apparatuses have been used in the extraction and separation of such biological substances. For the magnetic frame method, the principle is that firstly the substances to be separated and nano magnetic beads are combined in a liquid-filled container (such as a test tube) and react to form a complex, then a magnetic field is applied outside of the container to attract and adhere the magnetic beads to the wall, and then waste liquid is removed in some way using various different removal methods. The first drawback of this method is that an operator must mark each test tube, and then proceed to the next step. Time is needed to separate a sample, usually 30 minutes, and N samples need N*30 minutes. The second drawback is that the magnetic beads precipitate very quickly in the liquid, and to keep all the substances in the liquid in a uniform state, each operation process must be added with a mixing action, such as with spiral vortex device mixed processing, which increases the operation steps and greatly reduces the operating speed. The third drawback is that before each use of the vortex device, a test tube cover must be tightened, otherwise the operation steps cannot be completed. These drawbacks cannot keep pace with the technology and speed requirements of the modern biological/biotechnological field.

For the externally-applied magnetic field, it is generally adopted in the prior art that a magnet is either extended into the liquid containing the magnetic bead-separated substance complex or is put up against the outside wall of the container, so that the magnetic beads in the complex are gathered on one side of the container wail to facilitate waste liquid separation.

In the prior art method where the magnet extends into the complex directly, with multiple times of usage of the magnet, the surface of the magnet is very easily corroded, as many complex mixed liquids themselves are corrosive. Further, the magnetic force of the magnet may be damaged, and the service life of the magnet is thus severely affected.

For an automatic extraction apparatus, most existing automatic DNA extraction equipment on the market adopts the principle of the combination of immunology and nanometer magnetic beads to complete the substance separation process. The basic design principle is similar to the magnetic frame method, only adding an automatic control function and other heating functions. The first drawback of the automatic extraction apparatus is that the preparation stage is particularly complicated. The second drawback is that data input is very troublesome and before starting the program, repeated checks must be done. The third drawback is that if one step is incorrect, the whole batch of samples is ruined. The fourth drawback is that even after start-up, the automatic operation time is very long (usually 30 to 150 minutes, the reason is that dehydration is needed in some steps), and the existing equipment all adopt heating/drying, waiting for drying and other methods, which greatly prolongs the operation time.

Thus, although the magnetic frame method and the automatic DNA extraction method make up for certain deficiencies of the traditional method (such as substances separated according to biological activity), owing to the deficiencies of structure design and technological processes of existing equipment, the operation steps are difficult to perform and are time consuming.

In the field of biotechnology, achieving dehydration is an important problem that is difficult to solve in biological substances separation processes. Dehydration is a so-called “problem” because the biological substances separation processes are mostly performed under the condition of the existence of the liquid, such as separating lymphocytes and cancer cells from blood, separating some bacteria, viruses and other harmful substances from milk, and the like. The separated substances always more or less have unwanted liquid substances remained from upstream liquids. While in order to remove these excess liquid substances, routine laboratory methods often employ post-processing measures such as high temperature drying, room temperature volatilizing, time delay waiting, air floating (wind blowing) and the like, which clearly result in disadvantages such as time consuming, high effort, cumbersome in practice and the like.

For example, in the process of using the magnetic bead method to extract nucleic acids, the lysis solution is to lyse the cell, and after the magnetic bead adsorbs the released nucleic acids, it is necessary to separate the magnetic bead from the lysis solution. While presently in the prior art, a common practice is that the magnetic beads are gathered at one side of a container (provided with the lysis solution and the magnetic bead, typically a centrifuge tube) by an externally applied magnetic field; then a transfer liquid gun is used to suction the lysis solution, for achieving a separation of the magnetic bead and the lysis solution. The separated magnetic bead is further eluted, so as to achieve an extraction of the object nucleic acids.

However, in this extraction method, in the process of separating the lysis solution and the magnetic bead adsorbing the nucleic acids, a large number of liquids have remained in the gathered magnetic bead. Therefore, the solid-liquid separation is not complete, thereby reducing the purity of the nucleic acids subsequently extracted. If dehydration operations are further employed for the magnetic bead, such as air flowing (wind blowing), filter paper adsorbing and the like, on one hand, it causes time consuming, high effort and inconvenience to operate and on the other hand, it may also lead to a damage or loss of the adsorbed nucleic acids.

Thus, it is an object of the present invention to overcome the deficiencies of the prior art separation devices and methods.

Further and other objects of the present invention will become apparent to those skilled in the art from reading the following summary of the invention and the embodiments described and illustrated herein.

### SUMMARY OF THE INVENTION

In accordance with an aspect of the present invention, there is provided a multifunctional biological substance separation device comprising a housing, a spindle push rod running through the housing, a return device, a magnetic sleeve connecting ring and an electric actuator, wherein the two ends of the spindle push rod both protrude from the housing, and one end of the spindle push rod is provided with a magnetic column; the magnetic sleeve connecting ring is provided on the spindle push rod in a sleeving manner, and one end, which is positioned outside the housing, of the magnetic sleeve connecting ring is provided with a magnetic sleeve; the return device is provided in the housing, and is used for enabling the spindle push rod to move in a direction away from the magnetic sleeve; the electric actuator is connected to the magnetic sleeve connecting ring, and is used for enabling the magnetic sleeve connecting ring to rotate.

In an embodiment of the present invention, the device further comprises a snap-fit part fixedly provided in the housing; the return device comprising a first collar, a second collar and a first spring; the first collar, the first spring and the second collar are provided on the spindle push rod respectively in a sleeving manner, the first collar is abutted against the snap-fit part, the second collar is fixedly connected to the spindle push rod, and the two ends of the first spring are abutted against the first collar and the second collar respectively; one end, which is positioned outside the housing, of the spindle push rod is provided with a push rod wrapping sleeve; the closed end of the push rod wrapping sleeve wraps the end portion of the spindle push rod, and the open end is abutted against the second collar.

In an embodiment of the present invention, a positioning sleeve is provided in the housing, and the positioning sleeve is provided at the portion, which is positioned in the housing, of the push rod wrapping sleeve; and the inner wall of the positioning sleeve is fitted with the outer wall of the push rod wrapping sleeve.

In an embodiment of the present invention, the device further comprises a second spring and a magnetic sleeve disconnecting push rod provided on the housing, wherein one end of the second spring is fixed to the housing, the other end is abutted against one end of the magnetic sleeve disconnecting push rod, and the other end of the magnetic sleeve disconnecting push rod extends to an opening, through which the magnetic column runs, of the housing.

In an embodiment of the present invention, a circuit board is provided in the housing; the circuit board is electrically connected to the electric actuator, and the circuit board is also provided with a motor adjustment knob and a charging port that both protrude from the housing; and the housing is also provided with a button switch used for controlling a power source of the circuit board.

In an embodiment of the present invention, a handle is provided on the outside of the housing, one end of the handle is fixed to the housing, and the other end bends towards the direction of the magnetic column.

In an embodiment of the present invention, the electric actuator is provided in the housing, a driven gear is fixedly connected to the magnetic sleeve connecting ring, and the electric actuator is provided with a driving gear which engages with the driven gear.

In an embodiment of the present invention, the biological substance separation device also comprises a multi-head connecting bracket; the multi-head connecting bracket is connected to one end of the housing in a sleeving manner; the spindle push rod comprises a driving rod and a plurality of driven rods; all the driven rods are arranged side by side, and are fixed by a connecting rod, and the connecting rod is fixedly connected to the driving rod; the driving rod, the driven rods and the connecting rod are all provided on the multi-head connecting bracket; all the driven rods run through a fixing plate, and the ends, running through the fixing plate, of the driven rods are provided with magnetic columns, and the fixing plate is fixedly provided on the multi-head connecting bracket; a magnetic sleeve connecting ring is provided on each driven rod; one end of each magnetic sleeve connecting ring is provided with a magnetic sleeve, the other end is fixedly provided with a driven gear; every two adjacent driven gears engage with each other, and the electric actuator is provided on the multi-head connecting bracket, and is used for driving the driven gears to rotate.

In an embodiment of the present invention, the driving rod is parallel to all the driven rods, and the connecting rod is perpendicular to the driving rod.

In an embodiment of the present invention, the magnetic sleeve is a cavity with an open end and a closed end, wherein the open end is provided on the magnetic sleeve connecting ring in a sleeving manner, and the closed end is provided with a plurality of protrusions.

Accordingly, the present invention provides a multifunctional biological substance separation device comprising a housing, a spindle push rod running through the housing, a return device, a magnetic sleeve connecting ring and an electric actuator; the two ends of the spindle push rod both protrude from the housing, and one end of the spindle push rod is provided with a magnetic column; the magnetic sleeve connecting ring is provided on the spindle push rod in a sleeving manner, and one end, which is positioned outside the housing, of the magnetic sleeve connecting ring is provided with a magnetic sleeve; the return device is provided in the housing, and is used for enabling the spindle push rod to move in a direction away from the magnetic sleeve; and the electric actuator is connected to the magnetic sleeve connecting ring, and is used for enabling the magnetic sleeve connecting ring (comprising the magnetic sleeve provided thereon) to rotate. The multifunctional biological substance separation device provided by the present invention is particularly suitable for being applied to the process of nucleic acid extraction with a magnetic bead method; the multifunctional biological substance separation device has the effect of rapid separation of magnetic beads from the existence system (mostly lysate or eluent) of the magnetic beads; and the device is compact in structure, and easy and convenient to operate.

In accordance with an aspect of the present invention, there is provided a dehydration device comprising: a housing, a spindle pushrod, a magnetic sleeve and an electric actuator; wherein the spindle pushrod extends through the housing, with both ends of the spindle protruding from the housing, and the spindle pushrod can reciprocate back and forth along the housing, and one end of the spindle pushrod is provided with a magnetic column; the magnetic sleeve is sleeved on the magnetic column, and the magnetic column is positioned coaxially with the magnetic sleeve and located at the center of the magnetic sleeve, and one opened end of the magnetic sleeve is connected to the housing; and the electric actuator is provided within the housing and connected with the magnetic sleeve for rotating the magnetic sleeve.

In an embodiment of the present invention, the device further comprises a magnetic sleeve connecting ring provided on the housing, and one end of the magnetic sleeve connecting ring is sleeved with the magnetic sleeve, the other end is connected with the electric actuator.

In an embodiment of the present invention, the device further comprises a return device provided within the housing, and used for moving the spindle pushrod toward a direction away from the magnetic sleeve.

In an embodiment of the present invention, the device further comprises a circuit board provided within the housing; the electric actuator is electrically connected with the circuit board, and a motor adjustment knob and a charging port both protruding from the housing are also provided on the circuit board; and a push button switch for controlling the power supply of the circuit board is also provided on the housing.

In an embodiment of the present invention, one end of the magnetic sleeve is open, while the other end is closed, and the cross-section area of the magnetic sleeve is tapering along a direction from the open end to the closed end.

In an embodiment of the present invention, pluralities of protrusions are provided on an outer wall of one closed end of the magnetic sleeve.

In an embodiment of the present invention, the number of the protrusions is even, and the protrusions are symmetrically positioned in a direction along the axis of the magnetic sleeve.

In an embodiment of the present invention, the distances between each two adjacent protrusions are equal.

In accordance with an aspect of the present invention, there is provided a dehydration method using a dehydration device as described above, the method comprising: 1) pushing the spindle pushrod to allow the magnetic column to reach the closed end of the magnetic sleeve, and then stretching the magnetic sleeve of the dehydration device into a solid-liquid mixture containing magnetic substances; 2) adsorbing the magnetic substances to the closed end of the magnetic sleeve under the action of the magnetic column, and then removing the dehydration device from the solid-liquid mixture; and 3) controlling the electric actuator to allow the magnetic sleeve to rotate, and the magnetic substances are kept in adsorption state by the action of the magnetic field, while the moisture contained in the magnetic bead surface dehydrates under the action of centrifugal force.

In an embodiment of the present invention, in step 1), the solid-liquid mixture comprises a DNA lysis solution or elution buffer containing a magnetic bead.

In accordance with an aspect of the present invention, there is provided a magnetic sleeve, wherein the magnetic sleeve is a cavity with one closed end and one opened end; the cavity sequentially comprises a connecting sleeve and a body sleeve which are arranged integrally from the opened end to the closed end; a circle of convex ring is arranged on an edge of the end of the connecting sleeve.

In an embodiment of the present invention, impellers are arranged on an outer side of the closed end of the body sleeve.

In an embodiment of the present invention, the number of the impellers is an even number, and the impellers are symmetrically arranged in an axis direction of the body sleeve.

In an embodiment of the present invention, the cross sectional area of the body sleeve is gradually tapered in the direction from the connecting sleeve to the closed end.

In an embodiment of the present invention, the impellers extend from the closed end of the body sleeve to an external side wall of the body sleeve.

In an embodiment of the present invention, the connecting sleeve sequentially comprises a first lantern ring and a second lantern ring in the direction from the convex ring to the body sleeve; both the first lantern ring and the second lantern ring are gradually tapered in the direction from the convex ring to the body sleeve.

In an embodiment of the present invention, multiple reinforcing ribs are arranged on a side wall of the second lantern ring.

In an embodiment of the present invention, the reinforcing ribs surround the second lantern ring, and the distances among every two adjacent reinforcing ribs are equal.

In an embodiment of the present invention, an outer surface of the body sleeve is a roughed surface or a twilled surface.

In an embodiment of the present invention, multiple antiskid lugs are arranged on an inner side wall of the connecting sleeve.

Accordingly, the present invention provides a magnetic sleeve which is a cavity with one closed end and one opened end; the cavity sequentially comprises a connecting sleeve and a body sleeve which are arranged integrally from the opened end to the closed end; a circle of convex ring is arranged on the edge of the end of the connecting sleeve; in the usage process, the magnetic sleeve is in contact with a liquid mixing system containing magnetic beads, and the liquid mixing system does not submerge the connecting sleeve of the magnetic sleeve so that it can be ensured that a magnetic is not directly in contact with mixed liquid, thus the magnet is prevented from rusting, which prolongs its service life and improves effect. In addition, a circle of convex ring is arranged on the edge of the end of the connecting sleeve and mainly has fastening and shape fixing effect; specifically, due to the fact that the connecting sleeve is generally needed to be in sleeved connection with a certain position of the magnet or magnetic column, while the magnetic sleeve is generally made of a nonmagnetic material (such as plastic and rubber) and easily deform, the convex ring can have strength improving effect.

The present invention, in one embodiment, provides a multifunctional biological substance separation device that solves the technical problems of biological substance separation processes in the prior art, including processes adopting a magnetic bead method in the separation of cells, viruses, proteins, and nucleic acids. These prior art processes have the disadvantage of cumbersome operation processes, overabundant consumed time and energy, and poor separation efficiency.

The multifunctional biological substance separation device provided by the present invention is particularly suitable for being applied to the process of nucleic acid extraction with a magnetic bead method. The multifunctional biological substance separation device of the present invention has the effect of rapid separation of magnetic beads from the existence system (mostly lysate or eluent) of the magnetic beads; and the device is compact in structure, and easy and convenient to operate.

In order to achieve a purpose of the present invention, the following technical solution is, in one embodiment, adopted:

In one embodiment, the present invention provides a multifunctional biological substance separation device, comprising a housing, a spindle push rod running through the housing, a return device, a magnetic sleeve connecting ring and an electric actuator.

The two ends of the spindle push rod both protrude from the housing, and one end of the spindle push rod is provided with a magnetic column; the magnetic sleeve connecting ring is provided on the spindle push rod in a sleeving manner, and one end, which is positioned outside the housing, of the magnetic sleeve connecting ring is provided with a magnetic sleeve.

In an embodiment of the present invention, the spindle push rod and the magnetic sleeve connecting ring are not fixed relative to each other. The spindle push rod runs through the middle hollow part of the magnetic sleeve connecting ring sleeve to form a structure similar to the configuration that the magnetic sleeve connecting ring is provided on the spindle push rod in a sleeving manner.

The return device is provided in the housing, and is used for enabling the spindle push rod to move in a direction away from the magnetic sleeve; and the electric actuator is connected to the magnetic sleeve connecting ring and is used for enabling the magnetic sleeve connecting ring to rotate.

For the multifunctional biological substance separation device, the main structure thereof is the housing, wherein the housing is provided with the spindle push rod which runs through the housing; the two ends of the spindle push rod both protrude from the housing, and on one end of the spindle push rod is provided with a magnetic column. In addition, the spindle push rod is provided with the magnetic sleeve connecting ring and one end (positioned outside the housing) of the spindle push rod is provided with the magnetic sleeve. In this way, the magnetic sleeve connecting ring and the magnetic sleeve can rotate under the driving of the electric actuator; and when an acting force is applied to the end (without a magnetic column) of the spindle push rod, the relative movement can be achieved between the housing and the spindle push rod as well as the magnetic column thereof, and the spindle push rod can return to the initial position under the action of the return device. Namely, when the spindle push rod reciprocates in the housing, the magnetic column reciprocates in the housing as well. And the magnetic sleeve and the magnetic sleeve connecting ring rotate under the action of the electric actuator.

During the application of a biological substance separation device in accordance with one aspect of the present invention, such as applying the biological substance separation device to separation of magnetic bead lysates (containing the magnetic beads absorbing nucleic acid, and lysates); the magnetic sleeve is extended into the hybrid system and at the time, the spindle push rod is thrust to enable the end of the magnetic column to extend to the bottom of the magnetic sleeve; and the magnetic beads in the hybrid system are adhered to the magnetic sleeve under the action of the magnetic column and removed, thereby the rapid separation of the magnetic beads from the hybrid system is realized. Moreover, since the magnetic sleeve can rotate under the driving of the electric actuator, during the rotation, the magnetic beads retain on the magnetic sleeve under the action of the magnetic field, but the liquid is shed under the action of the centrifugal force to make further separation of the magnetic beads from the liquid. When the centrifugation is completed, the force on the spindle push rod is removed and the spindle push rod returns under the action of the return device to drive the magnetic column to go away from the bottom of the magnetic sleeve. The adhered magnetic beads are detached from the magnetic sleeve because of the loss of the adherence of the magnetic force, and the next step can be operated.

During the operation of a biological substance separation device of the present invention, the separation of the magnetic beads from the hybrid system can be realized. Accordingly, the disadvantages of the prior art such as, incomplete separation after the liquid phase is sucked out from the hybrid system, and the separation of the magnetic beads because of the easy sedimentation of the magnetic beads, can be overcome. Furthermore, after the magnetic beads are separated by the device, the further centrifugation operation can be realized to improve the effect of the overall biological substance separation. The disadvantage in the prior art methods of centrifugation, filtration, extraction, etc. of not being able to separate substances with the same mass but with different properties is overcome by the devices and methods of the present invention.

In the biological substance separation device of the present invention, in order to realize the effect of the rapid return of the spindle push rod, in one embodiment, a snap-fit part is fixedly provided in the housing; and the return device comprises a first collar, a second collar and a first spring; the first collar, the first spring and the second collar are provided on the spindle push rod respectively. The first collar is abutted against the snap-fit part; the second collar is fixedly connected to the spindle push rod; and the two ends of the first spring respectively are abutted against the first collar and the second collar; the end, which is positioned outside the housing is provided with a push rod wrapping sleeve; the closed end of the push rod wrapping sleeve wraps the end portion of the spindle push rod, and the open end is abutted against the second collar.

The snap-fit part is fixedly provided in the housing, so when the push rod wrapping sleeve is thrust, the spindle push rod and the second collar and other multiple structures all move along the housing (the magnetic column extends to the bottom of the magnetic sleeve); since the first collar is abutted against the snap-fit part and the snap-fit part is fixed, when the second collar moves toward the first collar, the first spring is compressed. If the applied compressive force is released now, since the second collar and the spindle push rod are fixed, after the acting force of the first spring is applied to the second collar, the spindle push rod is further driven to realize the return of the whole spindle push rod. The return manner is realized by employing the two collars provided on the spindle push rod in a sleeving manner. The collars have the effect of positioning of the spindle push rod, so the return effect is good and the spindle push rod Is not easily deflected.

In one embodiment of the present invention, a positioning sleeve is fixedly provided in the housing, and the positioning sleeve is provided on the portion, which is positioned in the housing, of the push rod wrapping sleeve; and the inner wall of the positioning sleeve is fitted with the outer wall of the push rod wrapping sleeve. The positioning sleeve locates the push rod wrapping sleeve and the housing, further make the spindle push rod stable and smooth in the reciprocation process.

In one embodiment of the present invention, a second spring and a magnetic sleeve disconnecting push rod provided on the housing are also comprised; one end of the second spring is fixed to the housing, the other end is abutted against one end of the magnetic sleeve disconnecting push rod, and the other end of the magnetic sleeve disconnecting push rod extends to the opening, through with the magnetic column run through, of the housing.

Under the external force, the magnetic sleeve disconnecting push rod moves along the housing, specifically toward the end, provided with the magnetic sleeve, of the housing. During the movement, the bottom of the magnetic sleeve disconnecting push can be abutted against the magnetic sleeve, and then the magnetic sleeve is enabled to be detached; and after the magnetic sleeve is detached, the magnetic sleeve disconnecting push rod returns to its initial position under the action of the second spring.

In one embodiment of the present invention, a circuit board is also provided in the housing; the circuit board is electrically connected to the electric actuator, and the circuit board is also provided with a motor adjustment knob and a charging port that both protrude from the housing; and the housing is also provided with a button switch used for controlling the power supply of the circuit board.

Integrated with all kinds of circuits, the circuit board can achieve output power adjustment of the electric actuator through the motor adjustment knob and directly control the rotation rate of the magnetic sleeve connecting ring as well as the rotation rate of the magnetic sleeve; the rotation speed regulation of the magnetic sleeve is achieved; charging effects of a power supply (provided in the housing) can be achieved through the charging ports (which can be arranged as USB interfaces for convenience); in addition, the housing is also provided with a button switch used for controlling the power supply of the circuit board.

In one embodiment of the present invention, a handle is provided on the outside of the housing, one end of the handle is fixed to the housing and the other end bends toward the magnetic column. Through the handle provided on the outside of the housing, on one hand, the biological substance separation device can be hung and placed successfully; and on the other hand, the handle is easy to grip and use because the handle is bent.

In one embodiment of the present invention, the electric actuator is provided in the housing, a driven gear is fixedly connected to the magnetic sleeve connecting ring and the electric actuator is provided with a driving gear engaged with the driven gear. The electric actuator (specifically arranged as a motor and can be, in one embodiment, arranged in the housing when the magnetic sleeve is single) is fixedly provided with the driving gear, after operation of the electric actuator, the driving gear rotates, further enabling the driven gear engaged therewith rotates, too, and ultimately driving a magnetic sleeve rotating device and the magnetic sleeve thereon to rotate; and the rotation of the magnetic sleeve can be achieved easily through this driving mode and the operation is convenient and feasible.

When a large number of samples need processing at one time, the mode of single head (i.e., each housing is provided with one magnetic sleeve) may not satisfy the need of batch processing, for which, in one embodiment of the present invention, a plurality of magnetic sleeves is provided. And it is necessary to simultaneously control the plurality of magnetic sleeves and magnetic columns through one spindle push rod. Therefore, in another embodiment of the present invention, the biological substance separation device comprises a multi-head connecting bracket; and the multi-head connecting bracket is connected to the housing in a sleeving manner; the spindle push rod comprises a driving rod and a plurality of driven rods; all the driven rods are arranged side by side and are fixed by a connecting rod, and the connecting rod is fixedly connected to the driving rod; the driving rod, the driven rods and the connecting rod are all provided on the multi-head connecting bracket; all the driven rods run through a fixing plate, the ends, running through the fixing plate, are provided with magnetic columns and the fixing plate is fixedly arranged on the multi-head connecting bracket; each driven rod is provided with a magnetic sleeve connecting ring; one end of each magnetic sleeve connecting ring is provided with a magnetic sleeve, and the other end is fixedly provided with a driven gear; every two adjacent driven gears are engaged with each other, and the electric actuator is provided on the multi-head connecting bracket and is used for driving the driven gears to rotate.

In the biological substance separation device comprising the “multi-head structure”, the spindle push rod may be inconsistent with that of “single-head structure”. In the “multi-head structure” embodiment, the spindle push rod may be divided into two parts, between which, the part in housing is the driving rod (i.e. the spindle push rod of “single-head structure”); and the plurality of driven rods are disposed outside the housing side by side; the plurality of driving rods are fixed by a connecting rod and are connected to the connecting rod. Then when the acting force is applied to the push rod wrapping sleeve, the driving rod pushes the connecting rod and all the driven rods to move while the magnetic column provided on the end portion of each driven rod is pushed and extends to the bottom of the corresponding magnetic sleeve.

Because each driven rod is provided with a magnetic sleeve connecting ring in a sleeving manner, one end of each magnetic sleeve connecting ring is provided with a magnetic sleeve and the other end is fixed with a gear, and every two adjacent driven gears are engaged with each other and the electric actuator is provided on the multi-head connecting bracket (easy for the rotation of a plurality of gears through one electric actuator). After the operation of the electric actuator, the rotation of all gear sets can be achieved as long as one of gears rotates.

Because of the structures of the multi-head connecting bracket and the fixing plate, when the spindle push rod (the driving rod) moves, only all the driven rods (comprising the magnetic columns on the ends) and the connecting rod move while the gear sets, the fixing plate and the multi-head connecting bracket are static relative to the housing.

In the “multi-head structure” biological substance separation device of the present invention, the corresponding structure in the housing is consistent with the single-head biological substance separation device. However, because of the plurality of magnetic sleeves (comprising magnetic columns) of this multi-head biological substance separation device, batch processing of treatment fluid with previous arrangement can be carried out, thus increasing processing efficiency, and saving a lot of time for experiments.

In one embodiment of the present invention, the driving rod is parallel to all the driven rods, and the connecting rod is perpendicular to the driving rod.

In one embodiment of the present invention, the magnetic sleeve is a cavity with an open end and a closed end, the open end is provided on the magnetic sleeve connecting ring in a sleeving manner, and a plurality of protrusions are provided at the closed end.

The arrangement of the plurality of protrusions outside the closed end of the magnetic sleeve, on one hand, facilitates the adsorption of magnetic beads on its surface, and on the other hand, effectively prevents the gathered magnetic beads from falling during the rotation process of the magnetic sleeve.

In one embodiment of the present invention, the outer surface of the blind end of the magnetic sleeve may be a V-shaped rough surface, a U-shaped rough surface, a V-shaped smooth surface or a U-shaped smooth surface. Also, the magnetic column may be cylindrical, square pillar-shaped, elliptic or pancake-shaped, or may be any shape suitable for being accommodated in a tubular container.

The closed end (blind end) of the magnetic sleeve may be V-shaped or may be U-shaped, and in order to improve the stirring effect, the blind end may be additionally provided with a linear or cross-shaped protrusion.

The magnetic sleeve may be made of plastic, rubber, glass or any known non-magnetic material, and may be transparent, non-transparent or colored. Also, the connection mode between the magnetic sleeve connecting ring and the electric actuator may vary and is not limited to rotation, that is, the magnetic sleeve may vibrate.

Compared with the prior art devices and methods, the present invention may have the following beneficial effects:

(1) The biological substance separation device provided by the present invention is advantageous in complete separation and high efficiency when being applied to the separation of biological substances; and during the process of separation using the device, the device can greatly simplify the operation, overcome the defects in the prior art that the magnetic beads are liable to be removed and the residual water in the magnetic beads is hard to remove;

(2) The device has a compact overall structure and is convenient to use, and when rotating, the magnetic sleeve can also take the stirring effect by adjusting the rotation speed thereof and has the function of a vortex device.

(3) Owing to the magnetic column, during the rotation of the magnetic sleeve, the magnetic beads retain on the magnetic sleeve under the action of the magnetic field, while the liquid is spun under the action of centrifugal force, thus further separating the magnetic beads from the liquid. In addition, the outer surface of the magnetic sleeve is rough, which takes the grinding effect on the mixing system during rotation, thus achieving the effect of breaking tissues and cells.

In accordance with another aspect of the present invention there is provided a dehydration device to solve the problems experienced by prior art biological substances separation techniques in that the solid-liquid separation of the magnetic bead containing separated substances is not complete (a technical problem that the liquid which is difficult to be removed is contained in the separated substances), and that the dehydration operation is time consuming and requires great effort. In the device of the present invention, detachment of a magnetic bead and the moisture contained on the surface thereof has been achieved by a combination of centripetal force of the magnetic column and centrifugal force produced during the rotation of the magnetic sleeve. The dehydration method of the present invention is more convenient and quicker than prior art dehydration methods such as general drying, wind blowing and the like, and will not cause damage to the separated substances.

In accordance with another aspect of the present invention there is provided a method of using the dehydration device of the present invention to dehydrate; the method is easy in operation, with high dehydration efficiency and short time.

In order to achieve a purpose of the dehydration device and method of the present invention, the following technical solution is, in one embodiment, adopted:

The present invention provides a dehydration device, and the dehydration device comprises a housing, a spindle pushrod, a magnetic sleeve and an electric actuator; wherein the spindle pushrod extends through the housing, with both ends of the spindle pushrod protruding from the housing, and the spindle pushrod can reciprocate back and forth along the housing, and one end of the spindle pushrod is provided with a magnetic column; the magnetic sleeve is sleeved on the magnetic column, and the magnetic column is positioned coaxially with the magnetic sleeve and located at the center of the magnetic sleeve, and one opened end of the magnetic sleeve is connected to the housing; and the electric actuator is provided within the housing and connected with the magnetic sleeve for rotating the magnetic sleeve.

For the device of the present invention, the main point lies in the interaction of magnetic centripetal force produced by the magnetic column and centrifugal force produced by the rotation of the magnetic sleeve, such that the position relation of the magnetic column and the magnetic sleeve is very important. In the device, the magnetic column is positioned at the center of the magnetic sleeve (the two are positioned coaxially, and no matter whether the magnetic column stretches into the closed end of the magnetic sleeve or not, the magnetic column is always located at the center position of the magnetic sleeve), furthermore providing a magnetic centripetal force to the magnetic bead of the closed end of the magnetic sleeve. When the centripetal force and the centrifugal force produced during the rotation of the magnetic sleeve are at a certain equilibrium point, the magnetic bead is left at the closed end of the magnetic sleeve by the action of the centripetal force, while the liquid dehydrates by the action of the centrifugal force, thereby the dehydration effect is achieved.

In particular, such dehydration device provided by the present invention has a body structure which is a housing, a spindle pushrod extends through the housing and reciprocates back and forth along the housing. Under the action of an external force, the magnetic column provided on the spindle pushrod can be stretched into the bottom of the magnetic sleeve (with a return device additionally provided, the magnetic column can be further returned, so as to achieve a reciprocal motion of the spindle pushrod and the magnetic bead); while the electric actuator provided within the housing can allow a rotation motion of the magnetic sleeve.

In the process of applying the dehydration device, for example it is used for separating the magnetic bead and the lysis solution (containing the magnetic bead adsorbing nucleic acids and the lysis solution); the magnetic sleeve is stretched into the mixture system, at this time pushing the spindle pushrod allows the end of the magnetic bead to stretch into the bottom of the magnetic sleeve; under the action of the magnetic bead, the magnetic bead in the mixture system are adsorbed onto the magnetic sleeve, and then removed, so as to achieve a fast separation of the magnetic bead and the mixture system. Further, as the magnetic sleeve can rotate by the drive of the electric actuator, such adsorbed magnetic bead can achieve a similar operation to the centrifugal dehydration (the centrifugal force adjustment can be achieved by controlling the rotation speed of the magnetic sleeve), such that the liquid phase contained in the interior of magnetic bead can be further separated. After centrifuging was completed, the force applied to the spindle pushrod is removed, and the spindle pushrod is returned under the action of the return device, driving the magnetic column away from the bottom of the magnetic sleeve. The adsorbed magnetic bead is detached from the upper magnetic sleeve due to the loss of the adsorption of the magnetic force, and it can be proceeded into the next operation.

It can be seen that, such dehydration device provided by the present invention largely simplifies the dehydration process, greatly shortens the dehydration time, and in the case of skillful operation, the dehydration process can be completed in a few seconds, and no damage will be caused to the separated substances in the dehydration process, In addition, this dehydration device has a compact structure, which is very convenient to operate under aseptic conditions.

Alternatively, a magnetic sleeve connecting ring is also included. The magnetic sleeve connecting ring is provided on the housing, and one end of the magnetic sleeve connecting ring is sleeved with the magnetic sleeve, the other end is connected with the electric actuator.

In the present invention, the magnetic sleeve connecting ring has a function of connecting the magnetic sleeve and the electric actuator; therefore, it is more convenient to achieve an effect that the electric actuator drives the magnetic sleeve to rotate.

Alternatively, a return device is also included. The return device is provided within the housing, and used for moving the spindle pushrod toward a direction away from the magnetic sleeve.

The effect of the return device is allowed to return the spindle pushrod, that is, the magnetic column is left from the bottom of the magnetic sleeve, so as to further improve the effect of the automation operation. For the specific structure of the return device, it is, in one embodiment, provided as a spring of which one end is fixed with the spindle pushrod, and the other end is fixed with the housing. With the return device and the pressure additionally applied to the spindle push rod, the reciprocal motion of the spindle pushrod (accompanied with the magnetic column) can be achieved.

Alternatively, a circuit board is also provided within the housing. The electric actuator is electrically connected with the circuit board, and a motor adjustment knob and a charging port both protruding from the housing are also provided on the circuit board; and a push button switch for controlling the power supply of the circuit board is also provided on the housing.

The circuit board can be integrated to various circuits for achieving an adjustment to the output power of the electric actuator by the motor adjustment knob, and directly controls the rotation speed rate of the magnetic sleeve connecting ring and the magnetic sleeve for achieving adjustment of the rotation speed of the magnetic sleeve; while with the charging port (for the sake of convenience, provided as a USB interface), it can achieve charging effect for the power supply (provided within the housing). In addition, a push bottom switch for controlling the power supply of the circuit board is also provided on the housing.

Alternatively, one end of the magnetic sleeve is opened, while other end is closed, and the cross-section area of the magnetic sleeve is tapering along a direction from the opened end to the closed end.

In the dehydration process, the magnetic sleeve is stretched into the mixed liquid system, and the outer wall of the magnetic sleeve is stained with water drop. Therefore, in order to improve the dehydration effect, the cross-section area of the magnetic sleeve is, in one embodiment, provided in a farm of tapering along a direction from the opened end to the closed end, and in a process of the rotational centrifuging, the magnetic sleeve having this structure facilitates the adhered water to detach from top to bottom, thereby to improve the dehydration efficiency.

Alternatively, a plurality of protrusions are provided on the outer wall of one closed end of the magnetic sleeve.

Alternatively, the number of the protrusions is even, and the protrusions are symmetrically positioned in a direction along the axis of the magnetic sleeve.

Alternatively, distances between the each two adjacent protrusions are equal.

In addition, in order to enhance a grinding effect of the magnetic sleeve in the rotating process, in one embodiment, the outer surface of the magnetic sleeve is configured to a rough face. In another embodiment, in order to enhance a stirring effect of the magnetic sleeve in the rotation process, a small wind blade with a straight line type or a cross type is provided at the blind end of the magnetic sleeve.

The method of using the dehydration device of the present invention to dehydrate comprises the steps as follows:

1) pushing the spindle pushrod to allow the magnetic column to reach the closed end of the magnetic sleeve, and then stretching the magnetic sleeve of the dehydration device into a solid-liquid mixture containing magnetic substances;

2) adsorbing the magnetic substances to the closed end of the magnetic sleeve under the action of the magnetic column, and then removing the dehydration device from the solid-liquid mixture;

3) controlling the electric actuator to allow the magnetic sleeve to rotate, and the magnetic substances are kept in adsorption state by the action of the magnetic field, while the moisture contained in the magnetic bead surface dehydrates under the action of the centrifugal force.

The dehydration method is performed by using a special dehydration device of the present invention, and for the solid-liquid mixture containing magnetic substances, it can achieve a separation of the magnetic bead and the liquid on the magnetic bead surface in a few seconds, and will not cause damage to the separated substances, which can greatly save time and improve the efficiency.

Alternatively, in the step 1), the solid-liquid mixture comprises a DNA lysis solution or elution buffer containing a magnetic bead.

For the DNA lysis solution or elution buffer containing a magnetic bead, it is very suitable to be a treatment object for this dehydration device. The magnetic bead can be all adsorbed and removed in the mixture system in very short time, and the rotational centrifuging process can also be completed in a few seconds. After magnetic force is released, the magnetic bead can be proceeded into the next operation process, and after eluted for again separating the magnetic bead, such that the desired nucleic acids can be obtained, while the magnetic bead will be otherwise stored or secondly used after centrifugal dehydration.

By using the above device, the present invention can solve problems which are difficult to solve in the prior art. When a nano magnetic bead to separate the biological substances is used, pushing the spindle pushrod of this device, the magnetic column can reach a magnetic field provided by the closed end of the magnetic sleeve. At this time, if the magnetic sleeve is stretched into the mixed liquid containing the magnetic bead, the magnetic bead can be adsorbed at the outside of the closed end of the magnetic sleeve. As the magnetic column is located at the center position of the magnetic sleeve, for the magnetic bead, the force provided by the magnetic column is a magnetic centripetal force. If the closed end of the magnetic sleeve is lifted out of liquid level, and a rotation knob is started, the magnetic sleeve is allowed to produce rotation motion, the rotation at this time will generates centrifugal force to the magnetic bead and the liquid on the surface thereof. As the magnetic bead is held in the same position under the action of the centripetal force of the magnetic field, the liquid dehydrates by the centrifugal force without effected by the magnetic force, and the interaction of the centrifugal force and the centripetal force functions as a separation effect of the magnetic bead and the liquid on the magnetic bead surface.

The whole dehydration process of the present invention can be completed in a few seconds.

Compared with the prior art, the beneficial effects of the present invention may comprise:

1) in this device, detachment of the moisture contained in the magnetic beam has been achieved by a combination of magnetic force of the magnetic column and centrifugal force produced during the rotation of the magnetic sleeve. It is more convenient and quicker than dehydration methods such as general drying, wind blowing and the like, and will not cause damage to the separated substances;

2) as the dehydration device has an impact and portable structure, it is very suitable for being an experimental tool in a field experiment, at the same time it is very convenient to operate under aseptic conditions;

3) further improvement on the outer surface of the magnetic sleeve (such as, configured to a rough face, increasing wind blades and the like) can allow the dehydration device to achieve effect of grinding and stirring device, with multiple functionalities, and can be a very suitable experimental tool in the field of biology.

Accordingly, the present invention provides a dehydration device comprising a housing, a spindle pushrod, a magnetic sleeve and an electric actuator; wherein the spindle pushrod extends through the housing, with both ends of the spindle pushrod protruding from the housing, and the spindle pushrod can reciprocate back and forth along the housing, and one end of the spindle pushrod is provided with a magnetic column; the magnetic sleeve is sleeved on the magnetic column, and the magnetic column is positioned coaxially with the magnetic sleeve and located at the center of the magnetic sleeve, and one opened end of the magnetic sleeve is connected to the housing; and the electric actuator is provided within the housing and connected with the magnetic sleeve for rotating the magnetic sleeve. In this device, detachment of a magnetic bead and the liquid contained on the surface thereof has been achieved by an interaction of centripetal force of the magnetic column and centrifugal force produced during the rotation of the magnetic sleeve. It is more convenient and quicker than dehydration methods such as general drying, wind blowing and the like, and will not cause damage to the separated substances.

In accordance with an aspect of the present invention, there is provided a magnetic sleeve to solve the technical problems in the prior art that the magnet serving as an externally-exerted magnetic field is easily corroded and the usage effect is affected.

In order to achieve a purpose of the magnetic sleeve of the present invention, the following technical solution is, in one embodiment, adopted:

The present invention provides a magnetic sleeve which is a cavity with one closed end and one opened end; the cavity sequentially comprises a connecting sleeve and a body sleeve which are arranged integrally from the opened end to the closed end; a circle of convex ring is arranged on an edge of the end of the connecting sleeve.

In an embodiment of the present invention, one end of the magnetic sleeve is closed, the other end of the magnetic sleeve is opened, and a connecting sleeve and a body sleeve are arranged sequentially from the opened end to the closed end. In the nucleic acid extraction process by a magnetic bead method, the magnetic sleeve can be arranged in a sleeving mode on the outer side of the magnet or magnetic column serving as the externally-exerted magnetic field, the connecting sleeve of the magnetic sleeve is fixed to the magnet or magnetic column or other parts, and the magnet or magnetic column is sleeved with the whole body sleeve portion. In the usage process, the magnetic sleeve is in contact with a liquid mixing system containing magnetic beads, and the liquid mixing system does not submerge the connecting sleeve of the magnetic sleeve so that it can be ensured that a magnet or magnetic column is not directly in contact with mixed liquid, thus the magnet or magnetic column is prevented from rusting, which prolongs its service life and improve effect. In addition, a circle of convex ring is arranged on the edge of the end of the connecting sleeve and mainly has fastening, shape fixing and strength improving effect; specifically, due to the fact that the connecting sleeve is usually needed to be in sleeved connection with a certain position of the magnet or magnetic column while the magnetic sleeve is generally made of plastic or rubber and is easily stretched, convex ring can have the shape fixing effect.

Optionally, impellers are arranged on the outer side of the closed end of the body sleeve.

In the usage process, the liquid mixing system possibly contains a lysis solution, magnetic beads and the like, the lysis solution can make target cells or tissues lyse and disrupt, and accordingly a target extract is released and is specifically adsorbed by the magnetic beads. However, for achieving a better adsorbing effect, the magnetic beads and the lysis solution are also needed to be mixed fully and evenly, therefore, the magnetic sleeve can be used for stirring, and the impellers are, in one embodiment, arranged at the closed end of the magnetic sleeve for improving stirring effect.

The magnetic sleeve with impeller structures can well stir a lysis solution mixing system and make the lysis solution mixing system mixed evenly, free nucleic acids can be completely adsorbed onto the magnetic beads as far as possible, in addition, all the used magnetic beads are stirred and are also adsorbed and gathered by the magnet or magnetic column, and the lysis and mixing waste solution is effectively adsorbed out.

Optionally, the number of the impellers is an even number, and the impellers are symmetrically arranged in an axis direction of the body sleeve.

Multiple impellers can be arranged, and for achieving even stirring effect, the number of the impellers is, in one embodiment, set as an even number, and all the impellers are symmetrically arranged in the axis direction of the body sleeve. In addition, the shapes and sizes of the impellers can be set optionally and are not specifically limited, for example, the impellers can be “−”-shaped and can be also “+”-shaped, and the shapes can be curved surfaces or planes. Moreover, the number of the impellers can be set as an odd number.

Optionally, the cross sectional area of the body sleeve is gradually tapered in the direction from the connecting sleeve to the closed end.

The body sleeve with a similar-cone-shaped structure is very conductive to natural falling of the liquid adhered to the surface of the body sleeve under the effect of gravitation, further conductive to the dehydration of the adsorbed magnetic beads and the decrease of waste liquid content and is conductive to smooth implementation of follow-up separation.

Optionally, the impellers extend from the closed end of the body sleeve to the external side wall of the body sleeve.

In addition, for the magnetic sleeve, the connecting relation between the magnetic sleeve and the magnet or magnetic column is indirect generally, for example, the magnetic sleeve can be fixed through one sleeve tube, the sleeve tube is arranged on the other driving part, and further the sleeve tube can perform rotary motion around the magnet or magnetic column. In the usage process, due to the fact that the magnet or magnetic column can adsorb the magnetic beads and further separate the magnetic beads and the waste liquid, and thus for further removing moisture contained in the separated magnetic beads, the magnetic sleeve can, in one embodiment, rotate and produce a centrifugal force so as to throw away waste liquid drops.

Moreover, the magnetic sleeve can rotate under the effect of the externally-added driving part so that the magnetic sleeve can have even stirring and mixing effect through its rotation after extending into the liquid mixing system, and in one embodiment, the impellers extend from the closed end of the body sleeve to the external side wall of the body sleeve for improving the stirring effect of the magnetic sleeve.

Optionally, the connecting sleeve sequentially comprises a first lantern ring and a second lantern ring in the direction from the convex ring to the body sleeve; both the first lantern ring and the second lantern ring are gradually tapered in the direction from the convex ring to the body sleeve.

The connecting sleeve mainly has the effect of connecting the magnetic sleeve to an externally-added connecting part, moreover, the externally-added driving part is needed to be in sleeved connection and fastened with the externally-added connecting part in the rotating process of the connecting sleeve. Therefore, for preventing the magnetic sleeve from falling, the connecting sleeve, in one embodiment, comprises the first lantern ring and the second lantern ring, both the first lantern ring and the second lantern ring are gradually tapered in the direction from the convex ring to the body sleeve. After the connecting sleeve with the two lantern ring structures and the gradually tapered cross.sectional area is in sleeved connection with the externally-added connecting part, the fastening-connecting effect can be achieved, and falling can be effectively prevented.

Optionally, multiple reinforcing ribs are arranged on the side wall of the second lantern ring.

Optionally, the reinforcing ribs surround the second lantern ring, and the distances among every two adjacent reinforcing ribs are equal.

The reinforcing ribs (in one embodiment, triangular reinforcing ribs) have the main effect of making the whole magnetic sleeve stable and balanced when the magnetic sleeve is placed (put upside down with an opening upward) and facilitating large-scale storage and transportation.

In addition, the reinforcing ribs also have the effect of improving the strength of the second lantern ring.

Optionally, the outer surface (generally the outer surface of the closed end) of the body sleeve is a roughed surface.

When the magnetic sleeve is driven to rotate by the externally-added driving part, it can not only have the stirring effect on the mixing system, but also have the effect similar to grinding according to the experimental demand, therefore the outer surface of the body sleeve is, in one embodiment, provided as a roughed surface, and the body sleeve with the roughed surface has very good grinding effect when being in contact with the liquid mixing system in the rotating process.

Optionally, multiple antiskid lugs are arranged on the inner side wall of the connecting sleeve.

For further improving the connecting fastness degree between the magnetic sleeve and the externally-added connecting part, in one embodiment, multiple antiskid lugs are arranged on the inner side wall of the connecting sleeve, and have the effect of increasing the friction force between the connecting part and the magnetic sleeve, so that the magnetic sleeve is tightly connected to the connecting part.

Furthermore, in one embodiment, the closed end (blind end) of the magnetic sleeve can be V-shaped or U-shaped, and the magnetic sleeve can be made of plastic, rubber, glass or any known material without magnetism for improving the stirring effect and can be transparent or non-transparent or be of different colors.

Compared with the prior art, the magnetic sleeve of the present invention may have the advantageous effects that:

1) in the usage process (for example, the DNA extraction process by utilizing a magnetic bead method), the magnetic sleeve extends into and is in contact with the liquid mixing system containing the magnetic beads, and the liquid mixing system does not submerge the end of the connecting sleeve of the magnetic sleeve, so that it can be ensured that the magnet or magnetic column is not directly in contact with the mixed liquid, the magnet or magnetic column is prevented from rusting, which prolongs its service life and improve effect, namely the magnetic sleeve has the effect similar to the effect of a protective sleeve on the externally-added magnet or magnetic column;

2) the magnetic sleeve shows a better stirring and grinding effect in the rotating process by adding the impellers on the outer wall of the magnetic sleeve or setting the surface of the magnetic sleeve to be the roughed surface and the like;

3) in addition, after the magnetic sleeve is connected to one driving part, the magnetic sleeve can perform rotary motion, which guarantee magnetic bead release, dehydration, tissue grinding and liquid stirring.

Further and other advantages of the present invention will be understood from the rest of the specification.

### BRIEF DESCRIPTION OF THE DRAWINGS

The present invention will be further understood from the following detailed description with references to the following drawings in which:

FIG. 1 is an opened perspective view of a multi-functional separation device having a single-head structure in accordance with an embodiment of the present invention.

FIG. 2 is a closed perspective view of the multi-functional separation device of FIG. 1.

FIG. 3 is a cross-sectional view of the multi-functional separation device of FIG. 1.

Reference numerals of FIGS. 1-3 include: Housing-101; Spindle push rod-102; Magnetic sleeve connecting ring-103; Electric actuator-104; Magnetic column-105; Magnetic sleeve-106; First collar-201; Second collar-202; First spring-203; Snap-fit part-204; Push rod wrapping sleeve-205; Positioning sleeve-206; Second spring-207; Magnetic sleeve disconnecting push rod-208; Button switch-209; Handle-210; Driven gear-211; and Protrusion/Impeller-305.

FIG. 4 is a front perspective view of a multi-functional separation device having a multi-head structure in accordance with an embodiment of the present invention.

FIG. 5 is a back perspective view of the multi-functional separation device having a multi-head structure of FIG. 4.

Reference numerals of FIGS. 4 and 5 include: Housing-101; Spindle push rod-102; Magnetic sleeve connecting ring-103; Electric actuator-104; Magnetic sleeve-106; Push rod wrapping sleeve-205; Positioning sleeve-206; Second spring-207; Magnetic sleeve disconnecting push rod-208; Button switch-209; Handle-210; Driven gear-211; Multi-head connecting bracket-301; Driven rod-302; Connecting rod-303; Fixing plate-304; and Protrusion/Impeller-305.

FIG. 6 is an opened perspective view of a dehydration device in accordance with an embodiment of the present invention.

FIG. 7 is a closed perspective view of the dehydration device of FIG. 6.

Reference numerals of FIGS. 6 and 7 include: Housing-101; Spindle pushrod-102; Magnetic sleeve connecting ring-103; Electric actuator-104; Magnetic column-105; Magnetic sleeve-106; Return device/First spring-203; Push rod wrapping sleeve-205; Push button switch-209; and Protrusion/Impeller-305.

FIG. 8 is a perspective view of a magnetic sleeve in accordance with an embodiment of the present invention.

FIG. 9 is a transparent perspective view of the magnetic sleeve of FIG. 8.

FIG. 10 is a side view of the magnetic sleeve of FIG. 8.

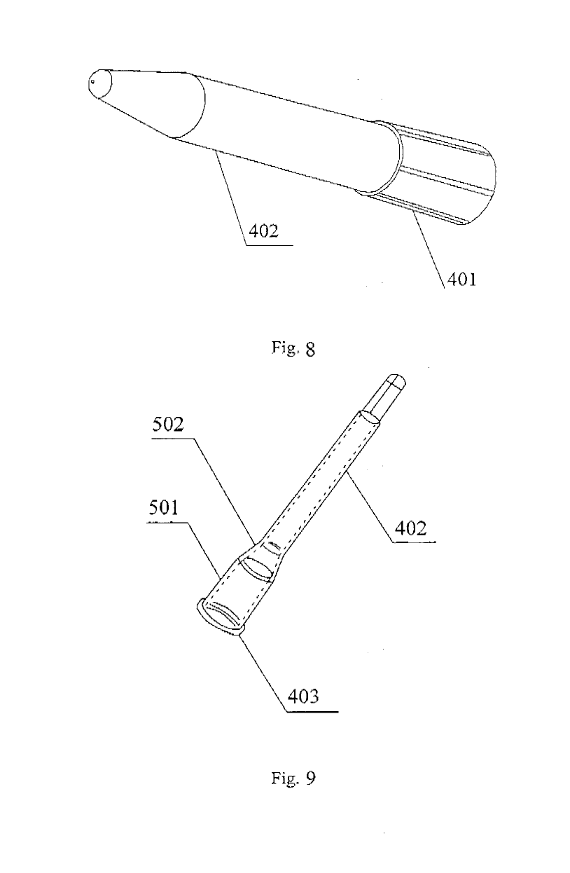

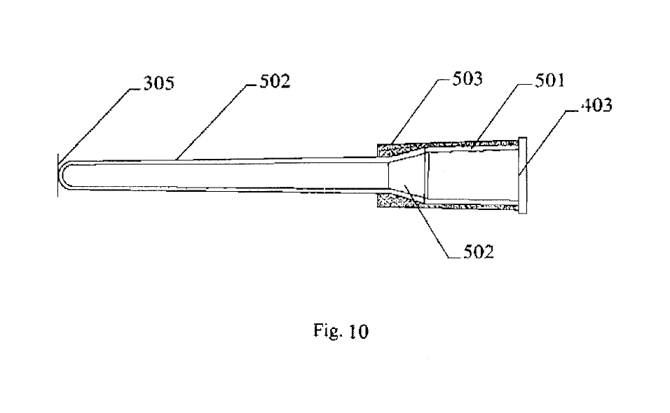

Reference numerals of FIGS. 8-10 include: Connecting sleeve-401; Body sleeve-402; Convex ring-403; First lantern ring-501; Second lantern ring-502; Reinforcing rib-503; Protrusion/Impeller-305.

Similar references are used in different figures to denote similar components.

### DETAILED DESCRIPTION OF THE INVENTION

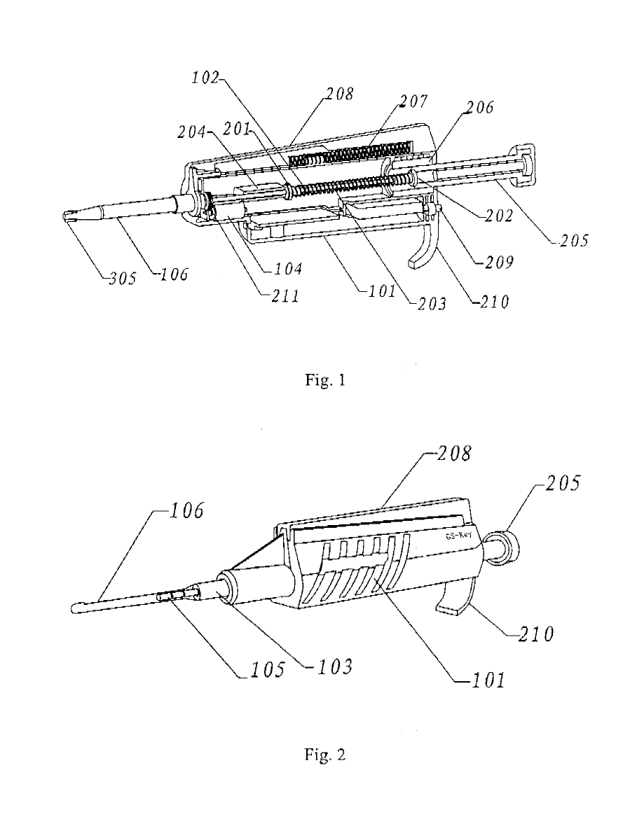

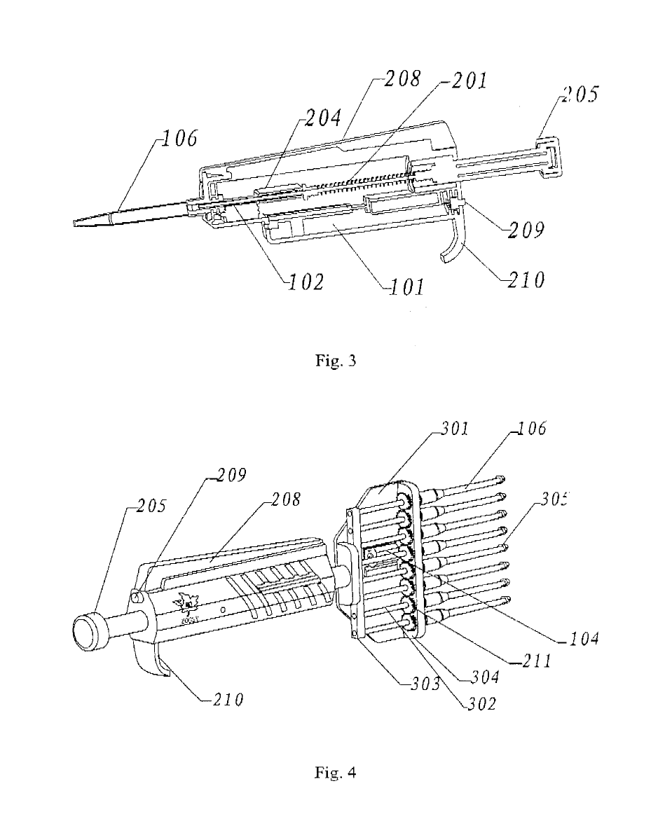

Referring now to FIGS. 1-3, the multifunctional biological substance separation device in accordance with an embodiment of the present invention comprises a housing 101, a spindle push rod 102 running through the housing 101, a return device, a magnetic sleeve connecting ring 103 and an electric actuator 104; the two ends of the spindle push rod both protrude from the housing 101, and one end of the spindle push rod is provided with a magnetic column 105 (shown in FIG. 2, the same below); the magnetic sleeve connecting ring 103 (not shown in FIGS. 1 and 3, shown in the corresponding position in FIG. 2, the same below) is provided on the spindle push rod 102 in a sleeving manner, and the end, which is positioned outside the housing 101, of the magnetic sleeve connecting ring 103 is provided with a magnetic sleeve 106; the return device is provided in the housing 101; and is used for enabling the spindle push rod 102 to move in a direction away from the magnetic sleeve 106; and the electric actuator 104 is connected to the magnetic sleeve connecting ring 103, and is used for enabling the magnetic sleeve connecting ring 103 to rotate.

The biological substance separation device of the present invention not only can achieve the separation effect of a mixing system (containing magnetic substances, such as magnetic beads) with high efficiency and rapidness, but also has the effects of a vortex device, a dehydration centrifuge, a grinding device and the like, and can be conveniently applied to biology experiments.

In an embodiment of the present invention, a snap-fit part 204 is fixedly provided in the housing 101; the return device comprises a first collar 201, a second collar 202 and a first spring 203;

the first collar 201, the first spring 203 and the second collar 202 are provided on the spindle push rod 102 respectively in a sleeving manner, the first collar 201 is abutted against the snap-fit part 204, the second collar 202 is fixedly connected to the spindle push rod 102, and the two ends of the first spring 203 are abutted against the first collar 201 and the second collar 202 respectively;

the end, which is positioned outside the housing 101, of the spindle push rod 102 is provided with a push rod wrapping sleeve 205; the closed end of the push rod wrapping sleeve 205 wraps the end of the spindle push rod 102, and the open end is abutted against the second collar 202.

For the snap-fit part 204, the structure is not particularly limited but configured to be fixed in the housing 101 and to have a larger contact surface to the first collar 201, in addition, in one embodiment, in order to achieve the effect of fixing the spindle push rod 102, the free end of the snap-fit part 204 is provided with a sliding chute, the spindle push rod 102 can run through the sliding chute to take the positioning effect on all of the spindle push rod 102, the first collar 201 and the second collar 202. Furthermore, in order to achieve steady connection between the first collar 201 and the second collar 202 with the first spring 203, both the first collar 201 and the second collar 202 comprise a protrusion for providing the spring (running through the two ends of the spring).

The return device with the above structure can rapidly and effectively achieve the effect of retuning the spindle push rod 102; the first spring 203 which is compressed can uniformly apply a force on the spindle push rod 102, to promote returning to the initial position.

In order to further ensure that the spindle push rod 102 is steady and smooth during reciprocating movement, on the basis of the solution, furthermore, a positioning sleeve 206 is also fixedly provided in the housing 101, and the positioning sleeve 206 is provided on the portion, which is positioned in the housing 101, of the push rod wrapping sleeve 205; and the inner wall of the positioning sleeve 206 is fitted with the outer wall of the push rod wrapping sleeve 205.

In the present invention, because the magnetic sleeve 106 is connected together with the magnetic sleeve connecting ring 103 in a sleeving manner, the magnetic sleeve 106 always needs to be taken down from the magnetic sleeve connecting ring 103 when being repeatedly used or meeting an operation requirement; in order to facilitate separating the magnetic sleeve 106 from the magnetic sleeve connecting ring 103, in one embodiment, a second spring 207 and a magnetic sleeve disconnecting push rod 208 which are provided on the housing 101 are also provided on the basis of the preferred solution; and one end of the second spring 207 is fixedly provided on the housing 101, the other end of the second spring 207 is abutted against one end of the magnetic sleeve disconnecting push rod 208, and the other end of the magnetic sleeve disconnecting push rod 208 extends to an opening, through which one end, provided with the magnetic column 105, of the spindle push rod 102, of the housing 101.

The magnetic sleeve disconnecting push rod 208 moves along the housing 101 under the action of an external force, in particular, the magnetic sleeve disconnecting push rod 208 moves towards the end, provided with the magnetic sleeve 106, of the housing 101, the bottom can be abutted against the magnetic sleeve 106 in the movement process, furthermore, the magnetic sleeve 106 can be separated, and the magnetic sleeve disconnecting push rod 208 returns to the original position under the action of the second spring 207 after the magnetic sleeve 106 is separated,

The structure of the magnetic sleeve disconnecting push rod 208 is not particularly limited and is only required to meet the requirement for reciprocating movement along the housing 101, for example, the magnetic sleeve disconnecting push rod 208 can have a groove-shaped structure, and the end, used for pushing the magnetic sleeve 106, of the magnetic sleeve disconnecting push rod 208 is also fixedly provided with a collar (embodied in the FIG. 3), and thus, the collar can be used for uniformly pushing the magnetic sleeve 106 from the magnetic sleeve connecting ring 103 when a pushing force is applied to the other end of the magnetic sleeve connecting ring 103. One end or part of the second spring 207 needs to be fixedly provided on the housing 101, and the other end of the second spring 207 is abutted against one end (any one of both ends) of the magnetic sleeve disconnecting push rod 208. In addition, in order to ensure that the structure is more compact and reasonable, the first spring 203 and the second spring 207 are parallel to each other.

In an embodiment of the present invention, the housing 101 is also internally provided with a circuit board (the mounting position is optional and is not shown in the figure); the circuit board is electrically connected to the electric actuator 104 and is also provided with motor regulation knobs and charging ports (not shown in the figure) which all protrude from the housing 101; and the housing 101 is also provided with a button switch 209 for controlling a power source of the circuit board.

Various circuits can be integrated through the circuit board, and the motor regulation knob can realize the output power regulation of the electric actuator 104 (to be specific, a motor) and directly controls the rotating speeds of the magnetic sleeve connecting ring 103 and the magnetic sleeve 106; the rotating speed regulation of the magnetic sleeve 106 is realized; and the effect of charging the power source can be achieved through the charging ports (which can be set to be USB interfaces for convenience); in addition, the housing 101 is also provided with the button switch 209 for controlling the power source of the circuit board. In the use process, the button switch 209 of the power source is turned on to enable the electric actuator 104 to enter a working state, and the working efficiency of the electric actuator 104 can be regulated by regulating the motor regulation knobs, It should be noted that the mounting position of the circuit board is optional, and the motor regulation knob is, in one embodiment, provided in the middle of the housing 101, so that the power of the motor is conveniently regulated in the use process; and the button switch 209 is provided at the end of the housing 101 so as to facilitate the pressing operation of the thumb.

In addition, in order to facilitate taking and placing biological substance separation device, on the basis of one embodiment, a handle 210 is provided outside the housing 101, one end of the handle 210 is fixedly provided on the housing 101, and the other end of the handle 210 is bent towards the magnetic column 105.

Under a general condition, each biological substance separation device is provided with one of the magnetic sleeves 106, namely a “single-head structure”; and the integrated structure is similar to the shape of a common pipette, in order to ensure that the magnetic sleeves 106 smoothly rotate, on the basis of one embodiment, the electric actuator 104 is provided in the housing 101, the magnetic sleeve connecting ring 103 is fixedly connected to the driven gear 211, and the electric actuator 104 is provided with a driving gear engaged with the driven gear 211.

By combining FIGS. 1 to 3, the technical solution embodies the structure and working principle of a single-end separation device, however, a single-end way (namely each housing 101 is provided with one of the magnetic sleeves 106) may not meet the batch processing requirement when a relatively large quantity of samples are processed at one time in the actual operation process, therefore, the plurality of magnetic sleeves 106 are preferably arranged, and it is necessary to simultaneously control the plurality of magnetic sleeves 106 and the magnetic column 105 by using one spindle push rod 102.

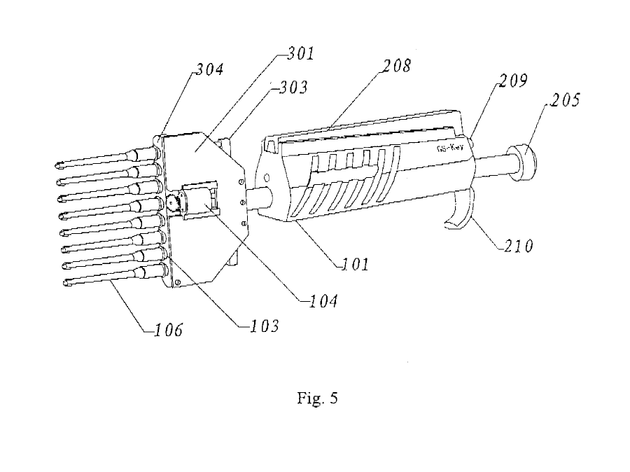

Therefore, with reference to FIGS. 4 to 5, the present invention also provides a biological substance separation device comprising a housing 101, a spindle push rod 102 running through the housing 101, a return device, a magnetic sleeve connecting ring 103 and an electric actuator 104.

Two ends of the spindle push rod 102 (particularly comprising a driving rod provided in the housing 101 and a driven rod 302 provided outside the housing 101 in the solution, wherein the driving rod is not shown in the figure because of being located in the housing 101) both protrude from the housing 101, and one end of the spindle push rod 102 is provided with the magnetic column 105; the magnetic sleeve connecting ring 103 is provided on the spindle push rod 102 in a sleeving manner (the structure of the magnetic sleeve connecting ring 103 is consistent with the structure shown in the FIGS. 1 to 3, and in the solution, the magnetic sleeve connecting ring 103 is particularly provided on the driven rod 302 in a sleeving manner and is not shown because being blocked in FIGS. 4 and 5), and the end, located outside the housing 101, of the magnetic sleeve connecting ring 103 is provided with the magnetic sleeves 106; the return device is provided in the housing 101 and is used for enabling the spindle push rod 102 to move far away from the magnetic sleeves 106; the electric actuator 104 is connected to the magnetic sleeve connecting ring 103 and is used for enabling the magnetic sleeve connecting ring 103 to rotate.

The positioning sleeve 206 is also fixedly provided in the housing 101, the positioning sleeve 206 is provided on the part, located in the housing 101, of the push rod wrapping sleeve 205; the inner wall of the positioning sleeve 206 is fitted with the outer wall of the push rod wrapping sleeve 205.

This device also comprises: a second spring 207 and a magnetic sleeve disconnecting push rod 208 provided on the housing 101; one end of the second spring 207 is fixed to the housing 101, the other end is abutted against the magnetic sleeve disconnecting push rod 208, and the other end of the magnetic sleeve disconnecting push rod 208 extends to an opening, through which one end, provided with the magnetic column 105, of the spindle push rod 102, of the housing 101.

The housing 101 is internally provided with a circuit board; the circuit board is electrically connected to the electric actuator 104, and the circuit board is also provided with a motor adjustment knob and a charging port which protrude from the housing 101; and the housing 101 is further provided with a button switch 209 for controlling the power source of the circuit board.

A handle 210 is provided on the outside of the housing 101, one end of the handle 210 is fixed to the housing 101, and the other end bends toward the direction of the magnetic column 105.

The biological substances separation device also comprises a multi-head connecting bracket 301; the multi-head connecting bracket 301 is connected to one end of the housing 101 in a sleeving manner; a spindle push rod 102 comprises a driving rod and a plurality of driven rods 302; all the driven rod 302 are arranged side by side, and are fixed by a connecting rod 303, the connecting rod 303 is fixedly connected to the driving rod; the driving rod, the driven rods 302 and the connecting rod 303 are arranged on the multi-head connecting bracket 301.

All the driven rods 302 run through a fixing plate 304, and one ends, running through the fixing plate 304, of the driven rods 302 are provided with magnetic columns 105, and the fixing plate 304 is fixedly provided on the multi-head connecting bracket 301.

Each driven rod 302 is provided with a magnetic sleeve connecting ring 103 in a sleeving manner; one end of each magnetic sleeve connecting ring 103 is provided with a magnetic sleeve 106, and the other end is fixedly provided with a gear; every two adjacent gears engage with each other, and the electric actuator 104 is arranged on the multi-head connecting bracket 301, and is used for driving the gear to rotate.

In the biological substance separation device with above “multi-head structure”, the spindle push rod 101 is divided into two parts, wherein the part in the housing 101 is a driving rod; the part outside of the housing 101 comprises a plurality of driven rods 302 arranged side by side; a plurality of driving rods 303 are fixed by the connecting rod 303, and the driving rod is fixedly connected to the connecting rod 303.

After applying the force on the push rod wrapping sleeve 205, the driving rod pushes the connecting rod 303 and all the driven rods 302 to move, at this time, the magnetic column 105 arranged at the end portion of each driven rod 302 is also pushed and extends to the bottom of the corresponding magnetic sleeve 106. In addition, as each driven rod 302 is provided with a magnetic sleeve connecting ring 103 in a sleeving manner; one end of each magnetic sleeve connecting ring 103 is provided with a magnetic sleeve 106, and the other end is fixedly provided with a gear; every two adjacent gear engage with each other, the electric actuator 104 (in particular, a motor) is provided on the multi-head connecting bracket 301 (which is helpful for realizing rotation of a plurality of gears by an electric actuator 104). After the operation of the electric actuator 104, the rotation of one gear among a plurality of gears can realize the rotation of all the gear sets, and further facilitate to realize the rotation of all of the magnetic sleeves 106.

As the structures of the multi-head connecting bracket 301 and the fixing plate 304, etc., when the spindle push rod 102 (in particular, a driving rod in the housing 101, in FIG. 5) moves, only all the driven rods 302 (which comprises the magnetic columns 105 at the end portions) and the connecting rod 303 move, and the gear set, the fixing plate 304, the multi-head connecting bracket 301 and the housing 101 are static relative to each other.

In the above biological substance separation device of above “multi-head structure”, the corresponding structure in the housing 101 is substantially consistent with the single-head biological substance separation device. However, because of the plurality of magnetic sleeves (comprising magnetic columns) of this multi-head biological substance separation device, batch processing of treatment fluid with previous arrangement can be carried out, thus increasing processing efficiency.

In one embodiment of the present invention, the driving rod is parallel to all the driven rods 302, and the connecting rod 303 is perpendicular to the driving rod; the magnetic sleeve 106 is a cavity with an open end and a closed end, and the open end is provided on the magnetic sleeve connecting ring 103 in a sleeving manner, and a plurality of projections/impellers 305 are arranged at the closed end, and the structure of the projections/impellers 305 is specifically an impeller to improve the function of stirring and scrolling In the process of rotation of the magnetic sleeve 106.

The biological substance separation device provided by the present invention opens a new way for the field of biological substance separation; and take the rapid separation of DNA as an example, there are only four steps, and the separation of DNA can be achieved within one minute.

The specific steps are as follows:

(1) adding magnetic beads, lysate and peripheral blood into a first tube, starting the rotation button, and accelerating the rotation with a device, and cells can be lysed instantly, and the DNA is released into the lysate;

(2) pressing the stop button of rotation, pushing the spindle push rod to provide a magnetic field, and under the function of the magnetic force of the magnetic columns, adhering the magnetic beads to the outside of the magnetic sleeve;

(3) washing the magnetic beads with DNA in lotions; and

(4) DNA and the magnetic beads are separated in the eluent, and providing the magnetic field, the magnetic beads are then scooped up, and the purified DNA can be obtained by disposing the magnetic beads.

It can be seen that the steps of biological substance separation are greatly simplified by this device, which builds up a milestone of the speed of extracting DNA (extract DNA in one minute), and creates a precedent that a single instrument can realize a plurality of functions in the biological substance separation field; specifically, it builds up a new method of dehydrated function in the separation substance project; establishes an example of using a miniature instrument to solve a big problem; builds up a new method of mixedly stirring the fluid in the biological separation process; embodies the value of exquisite combination of design and function of the magnetic sleeve (the back and forth movement and rotating movement existing in the magnetic field); and embodies the function of increasing the grinding and disrupting tissue after changing the shape of magnetic sleeve.

To sum up, the multifunctional biological substance separation device provided by the present invention is designed as a fast, convenient, agile and movable new instrument for fast removing magnetic particles of various sizes and shapes from a liquid medium.

The device is suitable for the removal of contaminants or other impurities from a contaminated liquid. For example, viruses, bacteria, toxins, hormones, drugs, insecticides, autoimmune proteins, antigens, haptens and so on are removed and purified from blood, bone marrow, cerebrospinal fluid, cell culture medium, food, milk and drink. Unwanted impurities can also be removed from biological reagents, industrial oils and chemical liquids. The device is mainly used for the rapid separation of biological substances in the field of medical biology. The common use of the device is that nucleic acids, proteins, viruses and a variety of cells are rapidly separated from different species of animal or plant tissues; the device can also be used for removing the harmful viruses (such as hepatitis, acquired immune deficiency syndrome), cancer cells (such as leukemia), autoimmune proteins (such as systemic lupus erythematosus, rheumatoid arthritis) and so on in the body to have the aim of treating and alleviating diseases.

In the separation aspect of biological substances in the field of medicine and biology, separation speed, accuracy, and agility of the multifunctional biological substance separation device of the present invention are all beyond the vast majority of the instruments in the conventional laboratory at present. The device of the present invention has the advantages of smart body, consolidation of function, convenient application, and simple operation, and sets up a milestone in the completion of various functions of a single model in the world of similar products; the multifunctional biological substance separation device has created the best in the world of similar products in terms of environmental protection, energy saving, safety and harmless, as well as the speed and accuracy of the separation of biological substances.

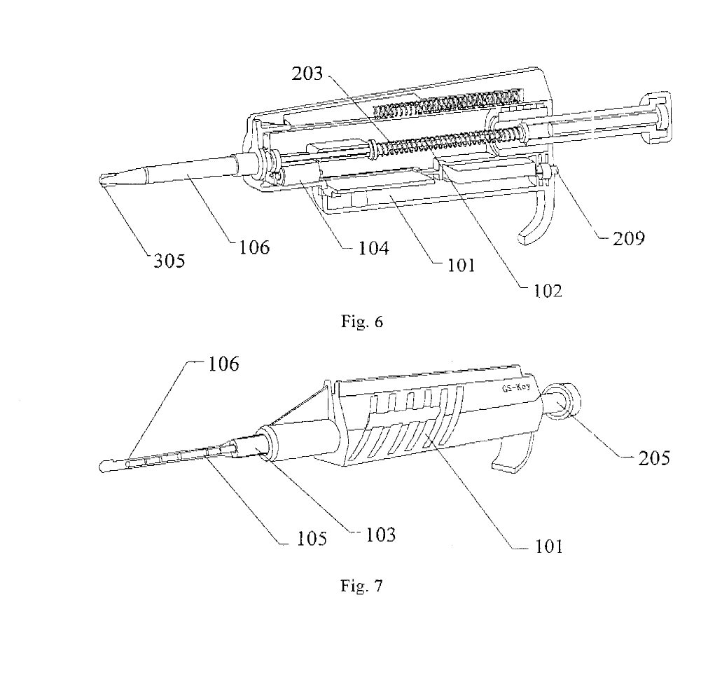

Referring now to FIGS. 6 and 7, such dehydration device provided by the present invention comprises a housing 101, a spindle pushrod 102, a magnetic sleeve 106 and an electric actuator 104.

The spindle pushrod 102 extends through the housing 101, with both ends of the spindle pushrod protruding from the housing 101, and the spindle pushrod 102 can reciprocate back and forth along the housing 101, and one end of the spindle pushrod 102 is provided with a magnetic column 105; the magnetic sleeve 106 is sleeved on the magnetic column 105 (particularly as shown in FIG. 7), and one opened end of the magnetic sleeve is connected to the housing 101; and the electric actuator 104 is provided within the housing 101 and connected with the magnetic sleeve 106 for rotating the magnetic sleeve 106.

Such dehydration device provided by the present invention has a body structure which is a housing 101, a spindle pushrod 102 extends through the housing 101 and reciprocates back and forth along the housing 101; under the action of an external force, the magnetic column 105 provided on the spindle pushrod 102 can be stretched into the bottom of the magnetic sleeve 106; while the electric actuator 104 provided within the housing 101 can rotate the magnetic sleeve 106.

In the process of applying the biological separation device, for example it is used for separating the magnetic bead and the lysis solution (containing the magnetic bead adsorbing nucleic acids and the lysis solution); the magnetic sleeve 106 is stretched into the mixture system, at this time pushing the spindle pushrod 102 allows the end of the magnetic column 105 stretch into the bottom of the magnetic sleeve 106; under the action of the magnetic column 105, the magnetic column 105 in the mixture system is adsorbed onto the magnetic sleeve 106, and then removed, so as to achieve a fast separation of the magnetic bead and the mixture system. Further, as the magnetic sleeve 106 can rotate by the drive of the electric actuator 104, the magnetic bead adsorbed on the outside of the closed end of the magnetic sleeve 106 can be remained on the magnetic sleeve 106 under the action of the magnetic force, while the liquid on the magnetic bead surface is separated with the magnetic bead under the action of the centrifugal force.

After centrifuging was completed, the force applied to the spindle pushrod 102 is removed, and the spindle pushrod 102 is returned under the action of the return device, in one embodiment a first spring 203, driving the magnetic column 105 away from the bottom of the magnetic sleeve 106. The adsorbed magnetic bead is detached from the upper magnetic sleeve 106 due to the loss of the adsorption of the magnetic force, and it can be very convenient to proceed into the next operation.

It can be seen that, such dehydration device provided by the present invention, which largely simplifies the dehydration process, greatly shortens the dehydration time, and the dehydration process can be completed in a few seconds, and no damage will be caused to the separated substances in the dehydration process. In addition, this dehydration device has a compact structure, which is very convenient to operate under aseptic conditions,

Based on the above technical schemes, in one embodiment, a magnetic sleeve connecting ring 201 is also comprised. The magnetic sleeve connecting ring 103 is provided on the housing 101, and one end of the magnetic sleeve connecting ring 103 is sleeved with the magnetic sleeve 106, the other end is connected with the electric actuator 104 (i.e., it is achieved that the magnetic sleeve 106 has connection with the electric actuator 104 by the magnetic sleeve connecting ring 103, and the electric actuator 104 allows rotation motion of the magnetic sleeve 106 by directly driving the magnetic sleeve connecting ring 103).

The magnetic sleeve connecting ring 103 has a function of connecting the magnetic sleeve 106 and the electric actuator 104, therefore it is more convenient to achieve an effect that the electric actuator 104 drives the magnetic sleeve 106 to rotate.

In the biological test, for various operating equipment, the high-low of its automation degree has direct effect on the success of the whole test and efficiency. Therefore, in order to improve the automation performance of the device of the present invention, based on the above schemes, in an embodiment, a return device 203, in one embodiment a first spring, is also included. The return device 203, is provided within the housing 101, and used for moving the spindle pushrod 102 toward a direction away from the magnetic sleeve 106. A circuit board is also provided within the housing 101. The electric actuator 104 is electrically connected with the circuit board, and a motor adjustment knob and a charging port both protruding from the housing 101 are also provided on the circuit board, A push button switch 209 for controlling the power supply of the circuit board is also provided on the housing 101.

The effect of the return device 203, is to return the spindle pushrod 102, that is, the magnetic column 105 is away from the bottom of the magnetic sleeve 106, to further improve the effect of the automation operation. With the return device 203 and a force additionally applied to the spindle pushrod 102, a reciprocation motion of the spindle pushrod 102 and the magnetic column 105 along the housing 101 can be achieved.

For the specific structure of the return device 202, in one embodiment it is provided as a spring of which one end is fixed with the spindle pushrod 102, and the other end is fixed with the housing 101.

In addition, it can also be a spring sleeved on the spindle pushrod 102, wherein one end is fixed with the spindle pushrod 102 by a connecting member, and other end is fixed with the housing 101.

The circuit board, motor adjustment knob and push button switch 209 can control the working state of the whole dehydration device, which is further convenient for use of the user.

In one embodiment provided by the present invention, the dehydration device comprises: a housing 101, a spindle pushrod 102, a magnetic sleeve 106 and an electric actuator 104, a magnetic sleeve connecting ring 103 and a return device 203.

The spindle pushrod 102 extends through the housing 101, with both ends of the spindle pushrod protruding from the housing 101, and the spindle pushrod 102 can reciprocate back and forth along the housing 101; one end of the spindle pushrod 102 is provided with a magnetic column 105; the magnetic sleeve 106 is sleeved on the magnetic column 105, and one opened end of the magnetic sleeve is connected to the housing 101; the electric actuator 104 is provided within the housing 101 and connected with the magnetic sleeve 106 for rotating the magnetic sleeve 106.

The magnetic sleeve connecting ring 103 is provided on the housing 101, and one end of the magnetic sleeve connecting ring 103 is sleeved with the magnetic sleeve 106, the other end is connected with the electric actuator 104; the return device 203 is provided within the housing 101, and used for moving the spindle pushrod 102 toward a direction away from the magnetic sleeve 106.

A circuit board is also provided within the housing 101, and the electric actuator 104 is electrically connected with the circuit board; a motor adjustment knob and a charging port both protruding from the housing 101 are also provided on the circuit board; and a push button switch 209 for controlling the power supply of the circuit board is also provided on the housing 101.

One end of the magnetic sleeve 106 is opened, while other end is closed, and the cross-section area of the magnetic sleeve 106 is tapering along a direction from the opened end to the closed end. A plurality of protrusions/impellers 305 are provided on the outer wall of one closed end of the magnetic sleeve 106. The number of the protrusions/impellers 305 is even, and the protrusions are symmetrically positioned in a direction along the axis of the magnetic sleeve 106. Distances between the each two adjacent protrusions/impellers 305 are equal. in the dehydration device with the above structures, it can be integrated with multiple functionalities such as watering, stirring, vortexing and the like, so as to meet the requirement of various biological tests. In addition, if this device is necessary to have a grinding function, correspondingly, the outer surface of the magnetic sleeve 106 is configured to a rough face, but for better stirring effect, a small wind blade with a straight line type or a cross type is additionally provided at the blind end of the magnetic sleeve 106.

In the present invention, a dehydration method of using the dehydration device is provided, particularly comprising the following steps:

S1: pushing the spindle pushrod 102 to allow the magnetic column 105 to reach the closed end of the magnetic sleeve 106, and then stretching the magnetic sleeve 106 of the dehydration device into a solid-liquid mixture containing magnetic substances.

In this step, the solid-liquid mixture comprises a DNA lysis solution or elution buffer containing a magnetic bead.

S2: adsorbing the magnetic substances to the closed end of the magnetic sleeve 106 under the action of the magnetic column 105, and then removing the dehydration device from the solid-liquid mixture.

In this step, an effect of directly removing the magnetic bead adsorbing separated substances from the mixed liquid system is achieved. Comparing this method with the traditional method of removing the liquid phase from the mixed liquid system, it has obvious advantages in that, firstly the liquid phase within the separated magnetic bead is remained less (in the operation process, it will be naturally freed under the gravity); additionally, the defect that the magnetic bead with the liquid phase removed may be lost in the prior art will be overcome.

S3: controlling the electric actuator 104 for rotating the magnetic sleeve 106, and the magnetic substances are kept in adsorption state by the action of the magnetic field, while the moisture contained in the magnetic bead surface dehydrates under the action of the centrifugal force.

In the method, based on the special dehydration device, directly removing the separated magnetic bead from the mixed liquid system, the dehydration effect is thus improved. In addition, in a process of rotation of the magnetic sleeve 106, the remained moisture may dehydrate, which further improves the dehydration effect. And the dehydration method can be performed by using a device, a combination of the device and the method can be complete the operation of dehydration in a few second with high efficiency, and no damage will be caused to the separated substances in the dehydration process. Thus, it has a very good application prospect.

Now referring to FIGS. 8-10, a magnetic sleeve in accordance with an embodiment of the present invention is illustrated. The magnetic sleeve 106, in one embodiment, is a cavity with one closed end and one opened end; the cavity sequentially comprises a connecting sleeve 401 and a body sleeve 402 which are arranged integrally from the opened end to the closed end; a circle of convex ring 403 is arranged on the edge of the end of the connecting sleeve 401.

In an embodiment of the present invention, one end of the magnetic sleeve is closed, the other end of the magnetic sleeve is opened, and the connecting sleeve 401 and the body sleeve 402 are arranged sequentially from the opened end to the closed end. In the nucleic acid extraction process of the magnetic bead method, the magnetic sleeve can be arranged in a sleeving mode on the outer side of the magnet or magnetic column serving as the externally-exerted magnetic field, the connecting sleeve 401 of the magnetic sleeve is fixed to the magnet or magnetic column, and the magnetic sleeve is sleeved with the whole body sleeve 402 portion. In the usage process, the magnetic sleeve is in contact with the liquid mixing system containing the magnetic beads, and the liquid mixing system does not submerge the connecting sleeve 401. of the magnetic sleeve, so that it can be ensured that the magnet or magnetic column is not directly in contact with the mixed liquid, the magnet or magnetic column is prevented from rusting, which prolongs its service life and improve effect. In addition, a circle of convex ring 403 is arranged on the edge of the end of the connecting sleeve 401 and mainly has the fastening and shape fixing effect; specifically, due to the fact that the connecting sleeve 401 is usually needed to be in sleeved connection with a certain position of the magnet or magnetic column while the magnetic sleeve is generally made of the nonmagnetic material and is easily stretched, convex ring 403 can have the shape fixing effect.

In one embodiment of the present invention, the magnetic sleeve is a cavity with one closed end and one opened end; the cavity sequentially comprises the connecting sleeve 401 and the body sleeve 402 which are arranged integrally from the opened end to the closed end; a circle of convex ring 403 is arranged on the edge of the end of the connecting sleeve 401. The protrusions/impellers 305 are arranged at the closed end of the body sleeve 402; the number of the protrusions/impellers 305 is an even number, and the impellers are symmetrically arranged in an axis direction of the body sleeve 402. The cross sectional area of the body sleeve 402 is gradually tapered in the direction from the connecting sleeve 401 to the closed end.

The magnetic sleeve with protrusions/impellers 305 structures can well stir a lysis solution mixing system and make the lysis solution mixing system mixed evenly, free nucleic acids can be completely adsorbed onto the magnetic beads as far as possible, in addition, all the used magnetic beads are stirred and are also adsorbed and gathered by the magnet or magnetic column, and the lysis and mixing waste solution is effectively adsorbed out, The body sleeve 402 with a similar-cone-shaped structure is very conductive to natural falling of the liquid adhered to the surface of the body sleeve under the effect of gravitation, further conductive to the dehydration of the adsorbed magnetic beads and the decrease of waste liquid content and is conductive to smooth implementation of follow-up separation.

In an embodiment of the present invention, the connecting sleeve 401 sequentially comprises a first lantern ring 501 and a second lantern ring 502 in the direction from the convex ring 403 to the body sleeve 402; both the first lantern ring 501 and the second lantern ring 502 are gradually tapered in the direction from the convex ring 403 to the body sleeve 402.

After the connecting sleeve 401 with the two lantern ring structures and the gradually tapered cross sectional area is in sleeved connection with the externally-added connecting part, the fastening-connecting effect can be achieved, and falling can be effectively prevented.

In an embodiment of the present invention, the magnetic sleeve is the cavity with one closed end and one opened end. The cavity sequentially comprises the connecting sleeve 401 and the body sleeve 402 which are arranged integrally from the opened end to the closed end. A circle of convex ring 403 is arranged on the edge of the end of the connecting sleeve 401. The protrusions/impellers 305 are arranged at the closed end of the body sleeve 402.

The number of the protrusions/impellers 305 is an even number, and the impellers are symmetrically arranged in the axis direction of the body sleeve 402. The cross sectional area of the body sleeve 402 is gradually tapered in the direction from the connecting sleeve 401 to the closed end. The protrusions/impellers 305 extend from the closed end of the body sleeve 402 to the external side wall of the body sleeve 402. The connecting sleeve 401 sequentially comprises a first lantern ring 501 and a second lantern ring 502 in the direction from the convex ring 403 to the body sleeve 402; both the first lantern ring 501 and the second lantern ring 502 are gradually tapered in the direction from the convex ring 403 to the body sleeve 402. Multiple reinforcing ribs 503 are arranged on the side wall of the second lantern ring 502; all the reinforcing ribs 503 surround the second lantern ring 502, and the distances among every two adjacent reinforcing ribs are equal.

It should be noted that with respect to the magnetic sleeve, the shapes and specific arrangement positions of the impellers 305 are not clearly shown in the provided attached drawings and are only given schematically.

In an embodiment of the present invention, the reinforcing ribs 503 have the main effect of making the whole magnetic sleeve stable and balanced when the magnetic sleeve is placed (put upside down with an opening upward) and facilitating large-scale storage and transportation. In addition, the reinforcing ribs 503 also have the effect of improving the strength of the second lantern ring 502 and further improve its usage effect.

In addition, for enabling the magnetic sleeve provided by the present invention to have grinding effect, specifically, when the magnetic sleeve is driven by the externally-added driving part to perform motion (generally rotation), the scheme provided with the reinforcing ribs 503 is further optimized, and the outer surface of the body sleeve 402 is set to be the roughed surface.

In the usage process, when the magnetic sleeve is driven by the externally-added driving part to perform rotation, the body sleeve 402 with the roughed or twilled surface (the roughed or twilled surface is mainly embodied at the closed end of the body sleeve) has very good grinding effect when being in contact with the liquid mixing system in the rotating process and can have good dispersing effect on tissues, cells and the like to be treated.

It should be noted that the preparation material adopted for the magnetic sleeve of the present invention is a non-magnetic material, such as plastic or rubber.

After the magnetic sleeve provided by the present invention is combined with the externally-added magnet or magnetic column, a control part capable of controlling the reciprocating motion of the magnet or magnetic column and the driving part capable of driving the magnet or magnetic column to rotate, a biological experimental device having very diversified functions can be formed through combination.

Specifically, the device can be suitable for removal of pollutants or other impurities from polluted liquids, for example, removal and purification of viruses, bacteria, toxins, hormones, medicines, pesticides, autoimmune proteins, antigens, haptens and the like from blood, bone marrows, cerebrospinal fluids, cell culture media, foods, milk and beverages, and removal of undesired impurities from biological reagents, industrial oil and chemical liquids. The device is mainly used for rapidly separating biological substances in the fields of medicine and biology. Commonly, the device rapidly separates nucleic acids, proteins, viruses and various cells from different kinds of animals or plant tissues, and can be also used for removing harmful viruses (such as hepatitis and Aids), cancer cells (such as leukemia), autoimmune proteins (such as systemic lupus erythematosus and rheumatoid arthritis) and the like so as to achieve the purpose of treating and relieving diseases.

The separation speed, accuracy and agility of the multifunctional bio-separation device surpass those of most of existing instruments for routine laboratories on the aspect of biological substance separation in the fields of medicine and biology. The ingenious device body, integrated functions, convenient application and simple operation of the device establish a milestone for achieving multiple functions through one single type in worldwide like products; the multifunctional bio-separation device takes the lead in the like products on the aspects of environmental protection, energy saving, safety, innocuousness and the speed and accuracy of separating biological substances.

In the description it should be noted that, unless otherwise specified and limited, the term “connection” and other terms should be understood generally, for example, they can be fixed connection, detachable connection, point connection, direct connection, indirect connection through a middle medium or internal communication between two elements, and the ordinary skilled in the art can understand the specific meanings of the above-mentioned terms in in specific circumstances.

While the foregoing provides a detailed description of embodiments of the present invention, it is to be understood that it is intended that all material contained herein be interpreted as illustrative of the present invention only and not in a limiting sense. Furthermore, numerous modifications, variations and adaptations may be made to the particular embodiments of the present invention described above without departing from the scope of the present invention, which is defined in the claims.

1. A multifunctional biological substance separation device comprising a housing, a spindle push rod running through the housing, a return device, a magnetic sleeve connecting ring and an electric actuator, wherein
the two ends of the spindle push rod both protrude from the housing, and one end of the spindle push rod is provided with a magnetic column; the magnetic sleeve connecting ring is provided on the spindle push rod in a sleeving manner, and one end, which is positioned outside the housing, of the magnetic sleeve connecting ring is provided with a magnetic sleeve;
the return device is provided in the housing, and is used for enabling the spindle push rod to move in a direction away from the magnetic sleeve; the electric actuator is connected to the magnetic sleeve connecting ring, and is used for enabling the magnetic sleeve connecting ring to rotate.

2. The multifunctional biological substance separation device according to claim 1, wherein a snap-fit part is fixedly provided in the housing; the return device comprises a first collar, a second collar and a first spring:
the first collar, the first spring and the second collar are provided on the spindle push rod respectively in a sleeving manner, the first collar is abutted against the snap-fit part, the second collar is fixedly connected to the spindle push rod, and the two ends of the first spring are abutted against the first collar and the second collar respectively;
one end, which is positioned outside the housing, of the spindle push rod is provided with a push rod wrapping sleeve; the closed end of the push rod wrapping sleeve wraps the end portion of the spindle push rod, and the open end is abutted against the second collar.

3. The multifunctional biological substance separation device according to claim 2, wherein a positioning sleeve is provided in the housing, and the positioning sleeve is provided at the portion, which is positioned in the housing, of the push rod wrapping sleeve; and the inner wall of the positioning sleeve is fitted with the outer wall of the push rod wrapping sleeve.

4. The multifunctional biological substance separation device according to claim 3, further comprising a second spring and a magnetic sleeve disconnecting push rod provided on the housing, wherein one end of the second spring is fixed to the housing, the other end is abutted against one end of the magnetic sleeve disconnecting push rod, and the other end of the magnetic sleeve disconnecting push rod extends to an opening, through which the magnetic column runs, of the housing.

5. The multifunctional biological substance separation device according to claim 4, wherein a circuit board is also provided in the housing; the circuit board is electrically connected to the electric actuator, and the circuit board is also provided with a motor adjustment knob and a charging port that both protrude from the housing; and the housing is also provided with a button switch used for controlling a power source of the circuit board.

6. The multifunctional biological substance separation device according to claim 5, wherein a handle is provided on the outside of the housing, one end of the handle is fixed to the housing, and the other end bends towards the direction of the magnetic column.

7. The multifunctional biological substance separation device according to claim 6, wherein:
the electric actuator is provided in the housing, a driven gear is fixedly connected to the magnetic sleeve connecting ring, and the electric actuator is provided with a driving gear which engages with the driven gear or
the biological substance separation device further comprises a multi-head connecting bracket; the multi-head connecting bracket is connected to one end of the housing in a sleeving manner;
the spindle push rod comprises a driving rod and a plurality of driven rods; all the driven rods are arranged side by side, and are fixed by a connecting rod, and the connecting rod is fixedly connected to the driving rod; the driving rod, the driven rods and the connecting rod are all provided on the multi-head connecting bracket;
all the driven rods run through a fixing plate, and the ends, running through the fixing plate, of the driven rods are provided with magnetic columns, and the fixing plate is fixedly provided on the multi-head connecting bracket;
a magnetic sleeve connecting ring is provided on each driven rod; one end of each magnetic sleeve connecting ring is provided with a magnetic sleeve, the other end is fixedly provided with a driven gear; every two adjacent driven gears engage with each other, and the electric actuator is provided on the multi-head connecting bracket, and is used for driving the driven gears to rotate and
optionally wherein the driving rod is parallel to all the driven rods, and the connecting rod is perpendicular to the driving rod.

8.-9. (canceled)

10. The multifunctional biological substance separation device according to claim 1, wherein the magnetic sleeve is a cavity with an open end and a closed end, wherein the open end is provided on the magnetic sleeve connecting ring in a sleeving manner, and the closed end is provided with a plurality of protrusions.

11. A dehydration device comprising: a housing, a spindle pushrod, a magnetic sleeve and an electric actuator;
wherein the spindle pushrod extends through the housing, with both ends of the spindle protruding from the housing, and the spindle pushrod can reciprocate back and forth along the housing, and one end of the spindle pushrod is provided with a magnetic column;
the magnetic sleeve is sleeved on the magnetic column, and the magnetic column is positioned coaxially with the magnetic sleeve and located at the center of the magnetic sleeve, and one opened end of the magnetic sleeve is connected to the housing; and
the electric actuator is provided within the housing and connected with the magnetic sleeve for rotating the magnetic sleeve.

12. The dehydration device according to claim 11, further comprising:
a magnetic sleeve connecting ring provided on the housing, and one end of the magnetic sleeve connecting ring is sleeved with the magnetic sleeve, the other end is connected with the electric actuator and/or
a return device provided within the housing, and used for moving the spindle pushrod toward a direction away from the magnetic sleeve.

13. (canceled)

14. The dehydration device according to claim 11, wherein a circuit board is also provided within the housing;
the electric actuator is electrically connected with the circuit board, and a motor adjustment knob and a charging port both protruding from the housing are also provided on the circuit board; and
a push button switch for controlling the power supply of the circuit board is also provided on the housing.

15. The dehydration device according to claim 14, wherein one end of the magnetic sleeve is opened, while the other end is closed, and the cross-section area of the magnetic sleeve is tapering along a direction from the opened end to the closed end.

16. The dehydration device according to claim 15, wherein pluralities of protrusions are provided on an outer wall of one closed end of the magnetic sleeve and optionally wherein the number of the protrusions is even, and the protrusions are symmetrically positioned in a direction along the axis of the magnetic sleeve and optionally wherein distances between each two adjacent protrusions are equal.

17.-18. (canceled)

19. A dehydration method of using the dehydration device according to claim 11, the method comprising:
1) pushing the spindle pushrod to allow the magnetic column to reach the closed end of the magnetic sleeve, and then stretching the magnetic sleeve of the dehydration device into a solid-liquid mixture containing magnetic substances;
2) adsorbing the magnetic substances to the closed end of the magnetic sleeve under the action of the magnetic column, and then removing the dehydration device from the solid-liquid mixture;
3) controlling the electric actuator to allow the magnetic sleeve to rotate, and the magnetic substances are kept in adsorption state by the action of the magnetic field, while the moisture contained in the magnetic bead surface dehydrates under the action of centrifugal force and
optionally wherein in step 1), the solid-liquid mixture comprises a DNA lysis solution or elution buffer containing a magnetic bead.

20. (canceled)

21. A magnetic sleeve, wherein the magnetic sleeve is a cavity with one closed end and one opened end; the cavity sequentially comprises a connecting sleeve and a body sleeve which are arranged integrally from the opened end to the closed end; a circle of convex ring is arranged on an edge of the end of the connecting sleeve.

22. The magnetic sleeve according to claim 21, wherein impellers are arranged on an outer side of the closed end of the body sleeve and optionally the number of the impellers is an even number, and the impellers are symmetrically arranged in an axis direction of the body sleeve.

23. (canceled)

24. The magnetic sleeve according to claim 22, wherein the cross sectional area of the body sleeve is gradually tapered in the direction from the connecting sleeve to the closed end and optionally wherein the impellers extend from the closed end of the body sleeve to an external side wall of the body sleeve.

25. (canceled)

26. The magnetic sleeve according to claim 21, wherein the connecting sleeve sequentially comprises a first lantern ring and a second lantern ring in the direction from the convex ring to the body sleeve; both the first lantern ring and the second lantern ring are gradually tapered in the direction from the convex ring to the body sleeve.

27. The magnetic sleeve according to claim 26, wherein:
multiple reinforcing ribs are arranged on a side wall of the second lantern ring or
an outer surface of the body sleeve is a roughed surface or a twilled surface or
multiple antiskid lugs are arranged on an inner side wall of the connecting sleeve.

28. The magnetic sleeve according to claim 27, wherein when multiple reinforcing ribs are arranged on a side wall of the second lantern ring, the reinforcing ribs surround the second lantern ring, and the distances among every two adjacent reinforcing ribs are equal.

29.-30. (canceled)
